(19) United States
(12) Patent Application Publication
(10) Pub. No.: US 2025/0282784 A1
(43) Pub. Date: Sep. 11, 2025

(54) CYTOCHROME BD OXIDASE INHIBITORS AND USES THEREOF

(71) Applicants: UNIVERSITY OF NOTRE DAME DU LAC, South Bend, IN (US); MONTANA STATE UNIVERSITY, Bozeman, MT (US); NANYANG TECHNOLOGICAL UNIVERSITY, Singapore (SG)

(72) Inventors: Marvin J. MILLER, South Bend, IN (US); Garrett C. MORASKI, Bozeman, MT (US); Kevin PETHE, Singapore (SG); Sashitherapany K, Singapore (SG)

(21) Appl. No.: 18/861,826

(22) PCT Filed: May 3, 2023
(86) PCT No.: PCT/US2023/066571

§ 371 Date: Oct. 30, 2024

Related U.S. Application Data
(60) Provisional application No. 63/337,836, filed on May 3, 2022.

(51) Int. Cl. C07D 487/04; A61K 31/498; A61K 31/519

(52) U.S. Cl. CPC C07D 487/04; A61K 31/498; A61K 31/519

(57) ABSTRACT

The present disclosure is directed, in part, to compounds, or pharmaceutically acceptable salts thereof, for modulating the activity of Cytochrome BD oxidase, or a mutant thereof. The disclosure also provides pharmaceutically acceptable compositions comprising compounds of the present disclosure and methods of using said compositions in the treatment of various diseases and disorders related to Cytochrome BD oxidase. Formula (I) and (II).

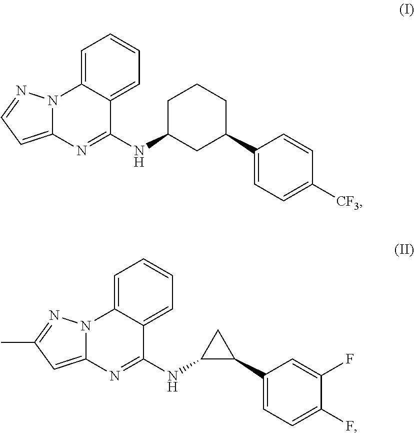 [SMILES: FC(F)(F)c1ccc([C@@H]2CCC[C@H](Nc3nc4ccnn4c4ccccc34)C2)cc1 ; Cc1cc2nc(N[C@@H]3C[C@H]3c3ccc(F)c(F)c3)c3ccccc3n2n1]

### CROSS-REFERENCE TO RELATED APPLICATIONS

This application claims priority to U.S. Provisional Application No. 63/337,836, filed May 3, 2022, which is incorporated herein by reference in its entirety.

### GOVERNMENT LICENSE RIGHTS

This invention was made with government support under Grant Number: R37 AI054193 awarded by the National Institutes of Health. The government has certain rights in the invention.

### TECHNICAL FIELD OF THE DISCLOSURE

The present disclosure is directed, in part, to compounds, or pharmaceutically acceptable salts thereof, for modulating the activity of Cytochrome BD oxidase or a mutant thereof. The disclosure also provides pharmaceutically acceptable compositions comprising compounds of the present disclosure and methods of using said compositions in the treatment of various diseases and disorders related to Cytochrome BD oxidase.

### BACKGROUND OF THE DISCLOSURE

Mycobacterial diseases have menaced humanity throughout the ages. Most notably, tuberculosis, caused by Mycobacterium tuberculosis (Mtb) is the leading cause of death worldwide from a single infectious agent, ranking above HIV/AIDS. Approximately 2 billion people are currently infected with tuberculosis. Though it remains latent in most people, about 10 million develop the active disease each year and more than 1.5 million die from TB each year. The situation is exacerbated by increasing numbers of cases associated with multidrug-resistant (MDR) and extensive drug-resistant (XDR) strains. After a nearly 40-year drought, a few new types of anti-TB drugs have been approved for use, but resistance to these new agents is already a clinical problem and new, more efficacious treatments are needed.

Even beyond this dire situation is the realization that non-tuberculosis mycobacterial (NTM) infections are on the rise. Most difficult to treat NTM infections are caused by the Mycobacterium avium complex (or MAC for short, which includes Mycobacterium avium and Mycobacterium intracellulare), and the Mycobacterium abscessus (differentiated into the three subspecies M. abscessus subsp. abscessus, M. abscessus subsp. massiliense, and M. abscessus subsp. bolletii) These NTM infections are exceptionally difficult to treat. Other examples of atypical pathogenic mycobacteria are Mycobacterium fortuitum and Mycobacterium kansasii.

The recently approved drug bedaquiline (Sirturo®) inhibits the F₁F₀ ATP synthase in Mtb and indicates that energy metabolism can be an effective target for development of anti-mycobacterial agents. Recently, a series of compounds have been reported that inhibit essential oxidative phosphorylation, including those that inhibit the terminal cytochrome bcc:aa₃ oxidase (Qcrb inhibitors) such as imidazopyridine carboxamides (G. C. Moraski et al., Advent of Imidazo[1,2-a]pyridine-3-carboxamides with Potent Multi- and Extended Drug Resistant Antituberculosis Activity. ACS Medicinal Chemistry Letters 2, 466-470 (2011); G. C. Moraski et al., Advancement of Imidazo[1,2-a]pyridines with Improved Pharmacokinetics and nM Activity vs. Mycobacterium tuberculosis. ACS Medicinal Chemistry Letters 4, 675-679 (2013); and K. A. Abrahams et al., Identification of Novel Imidazo[1,2-a]pyridine Inhibitors Targeting M. tuberculosis QcrB PLOS ONE 7, e52951 (2012)) like Q203 (Telecebec)(K. Pethe et al., Discovery of Q203, a potent clinical candidate for the treatment of tuberculosis. Nat Med 19, 1157-1160 (2013) that is now in phase 2 clinical trials. However, these compounds are bacteriostatic rather than bactericidal. The most probable reason is that functional redundancy between the cytochrome bcc:aa₃ and a second terminal oxidase, the cytochrome bd oxidase, protects M. tuberculosis from Q203-induced bacterial death, highlighting the attractiveness of the bd-type terminal oxidase for drug development (N. P. Kalia et al., Exploiting the synthetic lethality between terminal respiratory oxidases to kill Mycobacterium tuberculosis and clear host infection. Proc Natl Acad Sci USA 114, 7426-7431 (2017)). Thus, well-designed combinations of synergistic QerB and bd oxidase (BDO) inhibitors are of special interest.

Furthermore, observations made in the non-pathogenic mycobacteria Mycobacterium smegmatis revealed that genetic deletion of the BDO-encoding genes sensitized the bacteria to clofazimine, a second-line drug widely used to treat drug-resistant tuberculosis as well as NTM lung diseases (ref). Thus there is a need to identify a Cytochrome BD oxidase modulator for the treatment of these and other conditions. The present embodiments described herein fulfill these needs and others.

### SUMMARY OF EMBODIMENTS

The present disclosure provides compounds, or pharmaceutically acceptable salts thereof, in part, which modulate the activity of Cytochrome BD oxidase. The compounds can have, for example, a formula as described herein. In some embodiments, the compound is selected from a compound described herein. In some embodiments, methods of treating the diseases, disorders, and/or conditions, as described herein, are provided.

In some embodiments, the present disclosure provides compounds having a formula of Formula I, or pharmaceutically acceptable salts thereof:

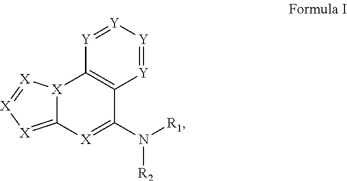

wherein the variables are as defined herein.

The present disclosure also provides pharmaceutical compositions comprising a compound, or a pharmaceutically acceptable salt thereof, as described herein. In some embodiments, the disclosure provides compositions comprising a compound of the present disclosure or a pharmaceutically acceptable derivative thereof and a pharmaceutically acceptable carrier, adjuvant, or vehicle.

The present disclosure also provides methods of using compounds of the present disclosure, pharmaceutically acceptable salts thereof, and pharmaceutically acceptable compositions thereof, for treating a variety of diseases, disorders, or conditions associated with regulating the activity of Cytochrome BD oxidase. In some embodiments, the disorder, disease, and/or condition is a mycobacterial infection. In some embodiments, the method further comprises administering an inhibitor of the oxidative phosphorylation processes in mycobacteria.

### BRIEF DESCRIPTION OF DRAWINGS

FIG. 1 illustrates that Compound 12 synergized with clofazimine to kill M. abscessus.

### DETAILED DESCRIPTION OF CERTAIN EMBODIMENTS

The disclosure may be more fully appreciated by reference to the following description, including the following definitions and examples. Certain features of the disclosed compositions and methods that are provided and described herein in the context of separate aspects may also be provided in combination in a single aspect. Alternatively, various features of the disclosed compositions and methods that are, for brevity, described in the context of a single aspect, may also be provided separately or in any subcombination.

At various places in the present specification, substituents of compounds are disclosed in groups or in ranges. It is specifically intended that the embodiments include each and every individual subcombination of the members of such groups and ranges. For example, the term “C₁₋₆ alkyl” or “C₁-C₆ alkyl” is specifically intended to individually disclose methyl, ethyl, C₃ alkyl, C₄ alkyl, C₅ alkyl, and C₆ alkyl.

It is further appreciated that certain embodiments, which are, for clarity, described in the context of separate embodiments, can also be provided in combination in a single embodiment. Conversely, various features of the embodiments, which are, for brevity, described in the context of a single embodiment, can also be provided separately or in any suitable subcombination. Additionally, for all purposes, the present disclosure encompasses not only the main group but also the main group absent one or more of the group members. The present disclosure, therefore, envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

All percentages and ratios used herein, unless otherwise indicated, are by weight.

Compounds of the present disclosure include those described generally herein and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N. Y., 2001.

The term “alkyl,” when used alone or as part of a substituent group, refers to a straight- or branched-chain hydrocarbon group, a spirocyclic group, or a fused or bridged bicyclic group, each of which has from 1 to 12 carbon atoms (“C₁-C₁₂”), preferably 1 to 6 carbons atoms (“C₁-C₆”), in the group Examples of alkyl groups include methyl (Me, C₁alkyl), ethyl (Et, C₂ alkyl), n-propyl (C₃ alkyl), isopropyl (C₃ alkyl), butyl (C₄ alkyl), isobutyl (C₄ alkyl), sec-butyl (C₄ alkyl), tert-butyl (C₄ alkyl), pentyl (C₅ alkyl), isopentyl (C₅ alkyl), tert-pentyl (C₅ alkyl), hexyl (C₆ alkyl), isohexyl (C₆ alkyl), and the like. The term “spirocyclic group” refers to spirocyclic compounds in which the two rings share only one single atom, the spiro atom, which is usually a quaternary carbon. Examples of spirocyclic compounds are spiro[2,3]undecane, spiro[3,3]heptane, and spiro[5,5]undecane. The term “fused bicyclic group” refers to fused bicyclic compounds, in which two rings share two adjacent atoms. Examples of fused bicyclic compounds include bicyclo[4.4.0]decane, α-thujene and decalin and the like. The term “bridged bicyclic group” refers to bridged bicyclic compounds, in which the two rings share three or more atoms, separating the two bridgehead atoms by a bridge containing at least one atom. Examples of bridged bicyclic compounds include bicyclo[2.2.1]heptane, bicyclo[1,1,1]pentane, bicyclo[2.2.1]heptane, bicyclo[2.2.2]octane, bicyclo[3.3.1]nonane, bicyclo[3.3.3]undecane, and the like. The term “haloalkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group, wherein one or more of the hydrogen atoms in the group have been replaced by a halogen atom. Examples of haloalkyl groups include trifluoromethyl (—CF₃, C1haloalkyl), trifluoroethyl (—CH₂CF₃, C₂haloalkyl), and the like.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups, and hybrids thereof such as (cycloalkyl)alkyl, (cycloalkenyl)alkyl, or (cycloalkyl)alkenyl.

As used herein, the term “bicyclic ring system” refers to any bicyclic ring system, i.e., carbocyclic or heterocyclic, saturated or having one or more units of unsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

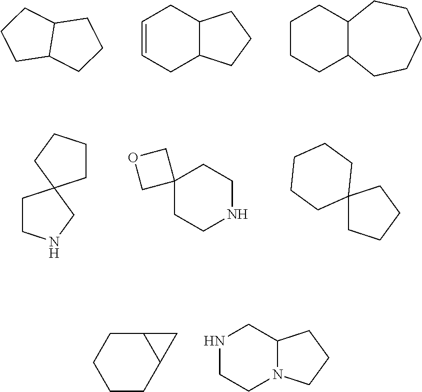

Exemplary bridged bicyclics include:

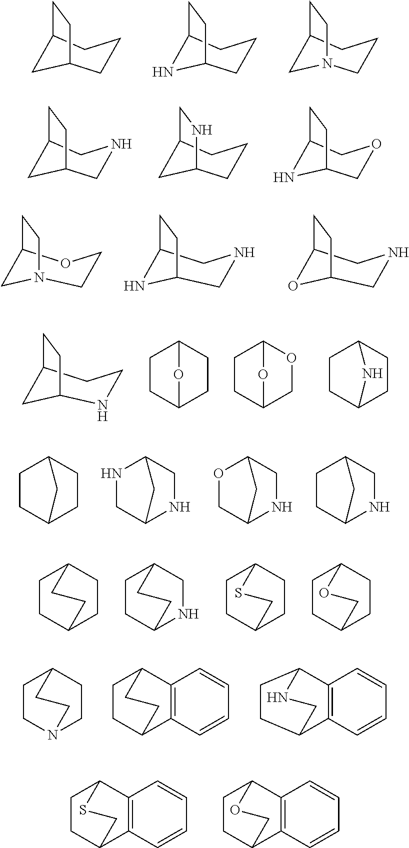

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including any oxidized form of nitrogen, sulfur, phosphorus, or silicon; the quaternized form of any basic nitrogen or, a substitutable nitrogen of a heterocyclic ring, for example, N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

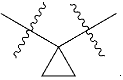 [SMILES: *C1(*)CC1]

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by the present disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. As used herein, “suitable substituent”, “substituent”, “optional substituent”, or the substituent for any optionally substituted group means a group that does not nullify the synthetic or pharmaceutical utility of the compounds described herein or the intermediates useful for preparing them. Examples of “suitable substituent”, “substituent”, “optional substituent”, or the substituent for any optionally substituted group includes, but are not limited to: C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈alkyl), —N(C₁-C₈ alkyl)₂, —NH (phenyl), —N(phenyl)₂, —CHO, —CO(C₁-C₆ alkyl), —CO(phenyl), —CO₂(C₁-C₆ alkyl), and —CO₂(phenyl). In some embodiments, one “suitable substituent”, “substituent”, or “optional substituent” is further substituted by one or more of C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈alkyl), —N(C₁-C₈ alkyl)₂, —NH (phenyl), —N(phenyl)₂, —CHO, —CO(C₁-C₆ alkyl), —CO(phenyl), —CO₂(C₁-C₆ alkyl), and —CO₂(phenyl). One of skill in art can readily choose a suitable substituent based on the stability and pharmacological and synthetic activity of the compounds described herein.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and references cited therein.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon, are within the scope of this disclosure. Such compounds are useful, for example, as analytical tools, as probes in biological assays, or as therapeutic agents in accordance with the present disclosure. In certain embodiments, a compound of the disclosure comprises one or more deuterium atoms.

As used herein, the term “inhibitor” is defined as a compound that binds to and/or inhibits Cytochrome BD oxidase, or a mutant thereof, with measurable affinity. In certain embodiments, an inhibitor has an IC₅₀ and/or binding constant of less than about 100 μM, less than about 50 μM, less than about 20 μM, less than about 10 μM, or less than about 5 μM.

The terms “measurable affinity” and “measurably inhibit,” as used herein, means a measurable change in Cytochrome BD oxidase, or a mutant thereof, activity between a sample comprising a compound of the present disclosure, or composition thereof, and Cytochrome BD oxidase, or a mutant thereof, and an equivalent sample comprising Cytochrome BD oxidase, or a mutant thereof, in the absence of said compound, or composition thereof.

The term “halo” or “halogen” refers to chloro, fluoro, bromo, or iodo.

The term “oxo” refers to an oxygen atom (i.e., =50) as a divalent substituent, forming a carbonyl group when attached to a carbon (e.g., C═O), or attached to a nitrogen or sulfur heteroatom forming a nitroso, sulfinyl, or sulfonyl.

The term “cycloalkyl” when used alone or as part of a substituent group refers to monocyclic, bicyclic, tricyclic, or poly cyclic non-aromatic hydrocarbon groups having from 3 to 22 carbon atoms (“C₃-C₂₂”), from 3 to 10 carbon atoms (“C₃-C₁₀”), from 3 to 6 carbon atoms (“C₃-C₆”), or from 3 to 7 carbon atoms (“C₃-C₇”). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), and the like. Suitable cycloalkyl groups include

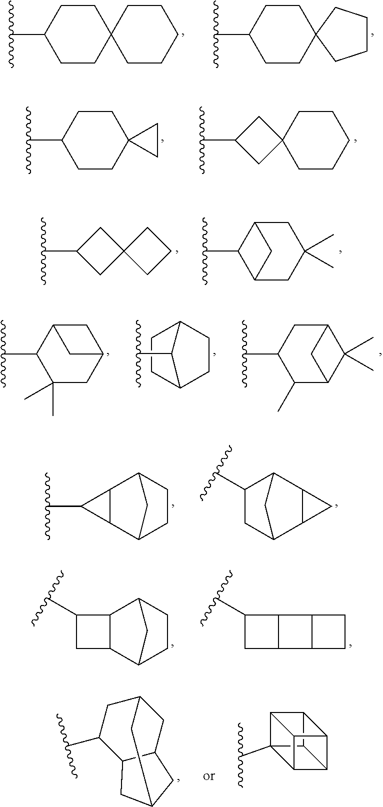

The term “heterocycloalkyl” when used alone or as part of a substituent group refers to any three to fourteen membered monocyclic, bicyclic, or tricyclic saturated ring structure containing at least one heteroatom selected from the group consisting of O, N, and S. Heterocycloalkyl groups may be described with respect to the number of atoms in the group, or with respect to the number of carbon atoms in the group.

The term “aryl” when used alone or as part of a substituent group, refers to a mono- or bicyclic-aromatic hydrocarbon ring structure having 6 or 10 carbon atoms in the ring system. Examples of aryl groups are phenyl and naphthyl.

The term “heteroaryl” when used alone or as part of a substituent group, refers to a mono-, bi-, or tricyclic-aromatic ring structure including carbon atoms as well as up to four heteroatoms selected from nitrogen, oxygen, and sulfur. Heteroaryl rings can include a total of 5, 6, 9, 10, or 14 ring atoms. Heteroaryl groups may be described with respect to the number of atoms in the group or with respect to the number of carbon atoms in the group. Thus, the term “5-14 membered heteroaryl” refers to a heteroaryl group containing between 5 and 14 ring atoms. The term —C₄-C₆ heteroaryl, for example, refers to a heteroaryl group containing four to six carbon atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like.

When a range of carbon atoms is used herein, for example, C₁-C₆ all ranges, as well as individual numbers of carbon atoms, are encompassed. For example, “C₁-C₃” includes C₁-C₃, C₁-C₂, C₂-C₃, C₁, C₂, and C₃. The range of carbon atoms may be expressed with alternative expressions. For example, the term “C₁-C₆” is an alternative expression of “C₁-C₆”.

When a ring system is described herein as having a range of members, for example, “5-14-membered”, all ranges, as well as individual numbers of atoms are encompassed. For example, “5-14-membered” includes 5-6-membered, 5-10-membered, 6-9-membered, 5-membered, 6-membered, 7-membered, 8-membered, and the like.

As used herein, “alkoxy” refers to an —O-alkyl group. Example alkoxy groups include methoxy, ethoxy, propoxy (e.g., n-propoxy and isopropoxy), t-butoxy, and the like.

The term “alkenyl” when used alone or as part of a substituent group refers to a straight- or branched-chain group having from 2 to 12 carbon atoms (“C₂-C₁₂”), preferably 2 to 6 carbons atoms (“C₂₋₆”), in the group, wherein the group includes at least one carbon-carbon double bond of alkenyl groups include vinyl (—CH═CH₂; C₂alkenyl), allyl (—CH₂—CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C4alkenyl), iso-butenyl (—CH═C(CH₃)₂; C₄ alkenyl), 2-butenyl (—CH₂CH═CHCH₃; C₄ alkyl), pentenyl (CH═CHCH₂CH₂CH₃ or CH₂═CHCH₂CH₂CH₂—; C₅ alkenyl), and the like.

The term “alkynyl” when used alone or as part of a substituent group refers to a straight- or branched-chain group having from 2 to 12 carbon atoms (“C₂-C₁₂”), preferably 2 to 6 carbons atoms (“C₂-C₆”), in the group, wherein the group includes at least one carbon-carbon triple bond. Examples of alkynyl groups include ethynyl (—C≡CH; C₂ alkynyl), propargyl (—CH₂—CH≡CH; C₃ alkynyl), and the like.

The compounds described herein can be asymmetric (e.g., having one or more stereocenters). All stereoisomers, such as enantiomers and diastereomers, are intended unless otherwise indicated. Compounds provided herein that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present embodiments. Geometric isomers of the compounds of the present embodiments are described and may be isolated as a mixture of isomers or as separated isomeric forms.

Compounds provided herein may also include tautomeric forms. All tautomeric forms are encompassed.

In some embodiments, the compounds may exist as rotational isomers. In some embodiments, the compounds exist as mixtures of rotational isomers in any proportion. In other embodiments, the compounds exist as particular rotational isomers, substantially free of other rotational isomers.

Compounds can also include all isotopes of atoms occurring in the intermediates or final compounds. Isotopes include those atoms having the same atomic number but different mass numbers. For example, isotopes of hydrogen include tritium and deuterium.

In some embodiments, the compounds, and salts thereof, are substantially isolated. By “substantially isolated” is meant that the compound is at least partially or substantially separated from the environment in which is formed or detected. Partial separation can include, for example, a composition enriched in the compound. Substantial separation can include compositions containing at least about 50%, at least about 60%, at least about 70%, at least about 80%, at least about 90%, at least about 95%, at least about 97%, or at least about 99% by weight of the compound, or salt thereof. Methods for isolating compounds and their salts are routine in the art.

Also provided herein are pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as, but not limited to, amines; alkali or organic salts of acidic residues such as, but not limited to, carboxylic acids; and the like. The pharmaceutically acceptable salts include, but are not limited to, the conventional non-toxic salts of the parent compound formed, for example, from non-toxic inorganic or organic acids. The pharmaceutically acceptable salts can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound provided herein with one or more solvent molecules.

“Subject” includes humans. The terms “human,” “patient,” and “subject” are used interchangeably herein.

As used herein, the phrase “in need thereof” means that the animal or mammal (subject) has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent. In some embodiments, the subject in need thereof is suspected of having the condition that needs to be treated.

As used herein, the term “synergy” or “synergistic effect” when used in connection with a description of the efficacy of a combination of agents or compounds, means any measured effect of the combination which is greater than the effect predicted from a sum of the effects of the individual agents or compounds. For example, as described herein, there are synergistic effects of treating a mycobacterial infection between (a) a QerB inhibitor and (b) an bd oxidase (BDO) inhibitor, which means that total inhibiting effects from the combination of (a) and (b) is greater than the sum of the inhibition effects of (a) and (b) alone.

As used herein, the term “mycobacterial infection” refers to a group of multisystem infections caused by the members of the family Mycobacteriaceae. Mycobacterium is a genus of Actinobacteria, given its own family, the Mycobacteriaceae. This genus includes pathogens known to cause serious diseases in mammals, including tuberculosis (Mycobacterium tuberculosis) and leprosy (Mycobacterium leprae) in humans.

As used herein, the phrase “integer from X to Y” means any integer that includes the endpoints. For example, the phrase “integer from X to Y” or “1-5” or “1 to 5” means 1, 2, 3, 4, or 5 or any value therein if not modified by the term “integer.”

“Compounds of the present disclosure,” “compounds as described herein” and equivalent expressions, are meant to embrace compounds of any formula or structural representation as described herein, as well as their subgenera, which expression includes the stereoisomers (e.g., enantiomers, diastereomers) and constitutional isomers (e.g., tautomers) of the various compounds and formula provided for herein as well as pharmaceutically acceptable salts or solvates thereof, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of the present disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well known in the art.

Throughout the description, where compositions are described as having, including, or comprising specific components, or where processes are described as having, including, or comprising specific process steps, it is contemplated that compositions described herein also consist essentially of, or consist of, the recited components, and that the processes described herein also consist essentially of, or consist of, the recited processing steps. Further, it should be understood that the order of steps or order for performing certain actions are immaterial so long as the process remains operable. Moreover, two or more steps or actions can be conducted simultaneously.

Compounds of the present disclosure, and pharmaceutical compositions thereof, are useful as inhibitors of Cytochrome BD oxidase or a mutant thereof. Without wishing to be bound by any particular theory, it is believed that compounds of the present disclosure, and pharmaceutical compositions thereof, may inhibit the activity of Cytochrome BD oxidase, or a mutant thereof, and thus treat certain diseases, disorders, or conditions associated with regulating the activity of Cytochrome BD oxidase, such as those described herein.

In some embodiments, provided are compounds having a formula of Formula I, or pharmaceutically acceptable salts thereof:

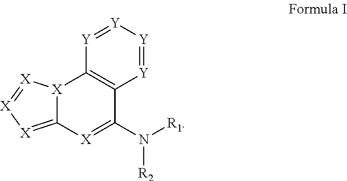

In some embodiments, the Formula I does not include the compound of

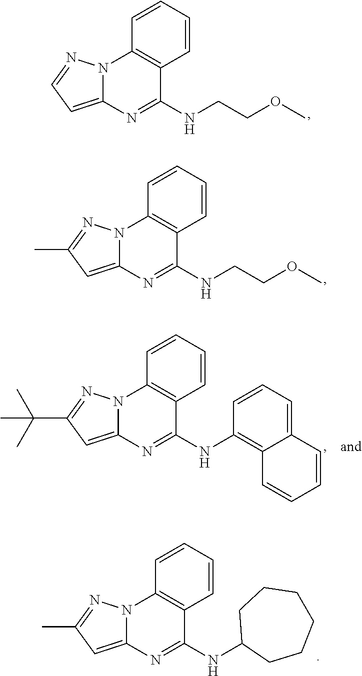

In some embodiments, the disclosure is directed to compounds of Formula I.

In some embodiments, the disclosure is directed to pharmaceutically acceptable salts or solvates of compounds of Formula I. In some embodiments, the disclosure is directed to pharmaceutically acceptable salts of compounds of Formula I. In some embodiments, the disclosure is directed to solvates of compounds of Formula I.

In some embodiments, each X is, independently, N or CR₃. In some embodiments, at least one X is N. In some embodiments, at least two Xs are N. In some embodiments, at least three Xs are N. In some embodiments, at least four Xs are N. In some embodiments, one X is N. In some embodiments, two Xs are N. In some embodiments, three Xs are N. In some embodiments, four Xs are N. In some embodiments, five Xs are N. In some embodiments, X is CR₃.

In some embodiments, each Y is, independently, N or CR₃. In some embodiments, at least one Y is N. In some embodiments, at least two Ys are N. In some embodiments, at least three Ys are N. In some embodiments, one Y is N. In some embodiments, two Ys are N. In some embodiments, three Ys are N. In some embodiments, four Ys are N. In some embodiments, five Ys are N. In some embodiments, Y is CR₄.

In some embodiments, R₁, R₂, R₃, and R₄ are each independently, H, D, halogen, R^{a}, —C(O)R^{a}, —CO₂R^{a}, —S(O)R^{a}, —SO₂R^{a}, —S(O)NHR^{a}, —SO₂NHR^{a}, —C(O)NHR^{a}, —N(R^{a})CO₂R³, or N(R^{a})CONR^{a}₂.

In some embodiments, R¹ is H, D, halogen, R^{a}, —C(O)R^{a}, —CO₂R^{a}, —S(O)R^{a}, —SO₂R^{a}, —S(O)NHR^{a}, —SO₂NHR^{a}, —C(O)NHR^{a}, —N(R^{a})CO₂R^{a}, or N(R^{a})CONR^{a}₂. In some embodiments, R₁ is H. In some embodiments, R¹ is D. In some embodiments, R₁ is halogen. In some embodiments, R₁ is R^{a}. In some embodiments, R₂ is —C(O)R^{a}. In some embodiments, R₁ is —CO₂R^{a}. In some embodiments, R₁ is —S(O)R^{a}. In some embodiments, R₁ is —SO₂R^{a}. In some embodiments, R₁ is —S(O)NHR^{a}. In some embodiments, R₁ is —SO₂NHR^{a}. In some embodiments, R₁ is —C(O)NHR^{a}. In some embodiments, R₁ is —N(R^{a})CO₂R^{a}. In some embodiments, R¹ is N(R^{a})CONR^{a}₂.

In some embodiments, R₂ is H, D, halogen, R^{a}, —C(O)R^{a}, —CO₂R², —S(O)R^{a}, —SO₂R^{a}, —S(O)NHR^{a}, —SO₂NHR^{a}, —C(O)NHR^{a}, —N(R^{a})CO₂R^{a}, or N(R^{a})CONR^{a}. In some embodiments, R₂ is H. In some embodiments, R₂ is D. In some embodiments, R₂ is halogen. In some embodiments, R₂ is R^{a}. In some embodiments, R₂ is —C(O)R^{a}. In some embodiments, R₂ is —CO₂R^{a}. In some embodiments, R₂ is —S(O)R^{a}. In some embodiments, R² is —SO₂R^{a}. In some embodiments, R₂ is —S(O)NHR^{a}. In some embodiments, R₂ is —SO₂NHR^{a}. In some embodiments, R₂ is —C(O)NHR^{a}. In some embodiments, R₂ is —N(R^{a})CO₂R^{a}. In some embodiments, R₂ is N(R^{a})CONR^{a}₂.

In some embodiments, R₃ is H, D, halogen, R^{a}, —C(O)R₃, —CO₂R₂, —S(O)R^{a}, —SO₂R^{a}, —S(O)NHR^{a}, —SO₂NHR^{a}, —C(O)NHR^{a}, —N(R^{a})CO₂R^{a}, or N(R^{a})CONR^{a}. In some embodiments, R₃ is H. In some embodiments, R₃ is D. In some embodiments, R^{a} is halogen. In some embodiments, R₃ is R^{a}. In some embodiments, R₃ is —C(O)R^{a}. In some embodiments, R₃ is —CO₂R^{a}. In some embodiments, R₃ is —S(O)R^{a}. In some embodiments, R₃ is —SO₂R^{a}. In some embodiments, R^{a} is —S(O)NHR^{a}. In some embodiments, R₃ is —SO₂NHR^{a}. In some embodiments, R₃ is —C(O)NHR^{a}. In some embodiments, R₃ is —N(R^{a})CO₂R^{a}. In some embodiments, R₃ is N(R^{a})CONR^{a}₂.

In some embodiments, R₄ is H, D, halogen, R^{a}, —C(O)R₃, —CO₃R^{a}, —S(O)R^{a}, —SO₂R^{a}, —S(O)NHR^{a}, —SO₂NHR^{a}, —C(O)NHR^{a}, —N(R^{a})CO₂R^{a}, or N(R^{a})CONR^{a}₂. In some embodiments, R₃ is H. In some embodiments, R^{a} is D. In some embodiments, R₃ is halogen. In some embodiments, R₃ is R^{a}. In some embodiments, R^{a} is —C(O)R^{a}. In some embodiments, R^{a} is —CO₂R^{a}. In some embodiments, R₃ is —S(O)R^{a}. In some embodiments, R₃ is —SO₂R^{a}. In some embodiments, R₄ is —S(O)NHR^{a}. In some embodiments, R₄ is —SO₂NHR^{a}. In some embodiments, R₄ is —C(O)NHR^{a}. In some embodiments, R₄ is —N(R^{a})CO₂R^{a}. In some embodiments, R₄ is N(R^{a})CONR^{a}₂.

In some embodiments, each R^{a} is independently H, D, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl. In some embodiments, each R^{a} is independently H. In some embodiments, each R^{a} is independently D. In some embodiments, each R^{a} is independently halogen. In some embodiments, each R^{a} is independently optionally substituted C₁-C₆ alkyl. In some embodiments, each R^{a} is independently C₁-C₆ alkoxyl. In some embodiments, each R^{a} is independently C₁-C₆ haloalkyl. In some embodiments, each R^{a} is independently optionally substituted C₃-C₂₂ cycloalkyl. In some embodiments, each R^{a} is independently C₄-C₁₀ heterocycle. In some embodiments, each R^{a} is independently C₆-C₁₀ aryl. In some embodiments, each R^{a} is independently or C₅-C₉ heteroaryl.

In some embodiments, each R^{a} is independently C₁-C₆ alkyl optionally substituted 1 to 3 times with R^{aa}. Each R^{aa} is independently selected from C₁-C₆ haloalkyl, aryl, wherein aryl can be further optionally substituted with heterocyclyl, C₁-C₆ haloalkyl, or C₂-C₁₀ alkenyl.

In some embodiments, each R^{a} is independently C₃-C₂₂ cycloalkyl optionally substituted 1 to 5 times with R^{aa}. Each R^{aa} is independently selected from halogen, C₁-C₆ alkyl, C₁-C₆ haloalkyl, aryl, wherein aryl can be further optionally substituted with heterocyclyl, C₁-C₆ haloalkyl, or C₂-C₁₀ alkenyl. In some embodiments, provided are compounds having a formula of

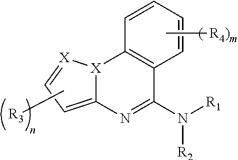

Formula II, or pharmaceutically acceptable salts or solvates thereof, wherein the variables are as defined herein. In some embodiments, n is 0-2 and m is 0-4. In some embodiments, n is 0. In some embodiments, n is 1. In some embodiments, n is 2. In some embodiments, m is 0. In some embodiments, m is 1. In some embodiments, m is 2. In some embodiments, m is 3. In some embodiments, m is 4. In some embodiments, R₂ is H.

In some embodiments, the disclosure is directed to compounds of Formula II.

In some embodiments, the disclosure is directed to pharmaceutically acceptable salts or solvates of compounds of Formula II. In some embodiments, provided are compounds having a formula of

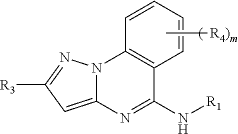

Formula III, or pharmaceutically acceptable salts or solvates thereof, wherein the variables are as defined herein. In some embodiments, m is 0-4. In some embodiments, m is 0. In some embodiments, m is 1. In some embodiments, m is 2. In some embodiments, m is 3. In some embodiments, m is 4. In some embodiments, R₂ is H.

In some embodiments, the disclosure is directed to compounds of Formula III. In some embodiments, provided are compounds having a formula of

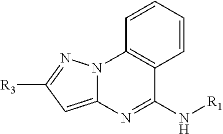

Formula IV, or pharmaceutically acceptable salts or solvates thereof, wherein the variables are as defined herein. In some embodiments, R₃ is H. In some embodiments, R₃ is optionally substituted C₁-C₆ alkyl. In some embodiments, the C₁-C₆ alkyl is optionally substituted with one or more of CF₃, halogen, C₁-C₆ alkoxy, SF₃, and SF₅. In some embodiments, the C₁-C₆ alkyl is optionally substituted with one or more of CF₃. In some embodiments, the C₁-C₆ alkyl is optionally substituted with one or more of halogen. In some embodiments, the C₁-C₆ alkyl is optionally substituted with one or more of C₁-C₆ alkoxy. In some embodiments, the C₁-C₆ alkyl is optionally substituted with one or more of SF₃. In some embodiments, the C₁-C₆ alkyl is optionally substituted with one or more of SF₅.

In some embodiments, the disclosure is directed to compounds of Formula IV.

In some embodiments, the disclosure is directed to pharmaceutically acceptable salts or solvates of compounds of Formula IV. In some embodiments, provided are compounds having a formula of

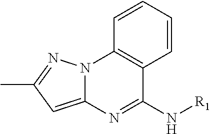

Formula V, or pharmaceutically acceptable salts or solvates thereof, wherein the variables are as defined herein. In some embodiments, R₁ is optionally substituted C₃-C₂₂ cycloalkyl.

In some embodiments, the disclosure is directed to compounds of Formula V.

In some embodiments, the disclosure is directed to pharmaceutically acceptable salts or solvates of compounds of Formula V.

In some embodiments, provided are compounds having a formula of

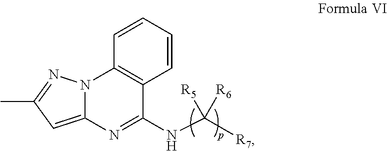

or pharmaceutically acceptable salts or solvates thereof, wherein the variables are as defined herein.

In some embodiments, R₅, R₆, and R₇ are each, independently, H, D, halogen, R^{b}, —C(O)R^{b}, —CO₂R^{b}, —S(O)R^{b}, SO₂R^{b}, —S(O)NHR^{b}, —SO₂NHR^{b}, —C(O)NHR^{b}, —N(R^{b})CO₂R^{b}, or N(R^{b})CONR^{b}.

In some embodiments, R₅ is H, D, halogen, R^{b}, —C(O)R^{b}, —CO₂R^{b}, —S(O)R^{b}, SO₂R^{b}, —S(O)NHR^{b}, —SO₂NHR^{b}, —C(O)NHR^{b}, —N(R^{b})CO₂R^{b}, or N(R^{b})CONR^{b}. In some embodiments, R₅ is H. In some embodiments, R₅ is D. In some embodiments, R₅ is halogen. In some embodiments, R₅ is R^{b}. In some embodiments, R₅ is —C(O)R^{b}. In some embodiments, R₅ is —CO₂R^{b}. In some embodiments, R₅ is —S(O)R^{b}. In some embodiments, R₅ is SO₂R^{b}. In some embodiments, R₅ is —S(O)NHR^{b}. In some embodiments, R₅ is —SO₂NHR^{b}. In some embodiments, R₅ is —C(O)NHR^{b}. In some embodiments, R₅ is —N(R^{b})CO₂R^{b}. In some embodiments, R₅ is or N(R^{b})CONR^{b}.

In some embodiments, R₆ is H, D, halogen, R^{b}, —C(O)R^{b}, —CO₂R^{b}, —S(O)R^{b}, SOR^{b}, —S(O)NHR^{b}, —SO₂NHR^{b}, —C(O)NHR^{b}, —N(R^{b})CO₂R^{b}, or N(R^{b})CONR^{b}. In some embodiments, R₆ is H. In some embodiments, R₆ is D. In some embodiments, R₆ is halogen. In some embodiments, R₆ is R^{b}. In some embodiments, R₆ is —C(O)R^{b}. In some embodiments, R₆ is —CO₂R^{b}. In some embodiments, R₆ is —S(O)R^{b}. In some embodiments, R₆ is SO₂R^{b}. In some embodiments, R₆ is —S(O)NHR^{b}. In some embodiments, R₆ is —SO₂NHR^{b}. In some embodiments, R₆ is —C(O)NHR^{b}. In some embodiments, R₆ is —N(R^{b})CO₂R^{b}. In some embodiments, R₆ is or N(R^{b})CONR^{b}.

In some embodiments, R₇ is H, D, halogen, R^{b}, —C(O)R^{b}, —CO₂R^{b}, —S(O)R^{b}, SO₂R^{b}, —S(O)NHR^{b}, —SO₂NHR^{b}, —C(O)NHR^{b}, —N(R^{b})CO₂R^{b}, or N(R^{b})CONR^{b}. In some embodiments, R₇ is H. In some embodiments, R₇ is D. In some embodiments, R₇ is halogen. In some embodiments, R₇ is R^{b}. In some embodiments, R₇ is —C(O)R^{b}. In some embodiments, R₇ is —CO₂R^{b}. In some embodiments, R₇ is —S(O)R^{b}. In some embodiments, R₇ is SO₂R^{b}. In some embodiments, R₇ is —S(O)NHR^{b}. In some embodiments, R₇ is —SO₂NHR^{b}. In some embodiments, R₇ is —C(O)NHR^{b}. In some embodiments, R₇ is —N(R^{b})CO₂R^{b}. In some embodiments, R₇ is or N(R^{b})CONR^{b}.

In some embodiments, each R^{b} is independently H, D, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl. In some embodiments, each R^{b} is independently H. In some embodiments, each R^{b} is independently D. In some embodiments, each R^{b} is independently halogen. In some embodiments, each R^{b} is independently optionally substituted C₁-C₆ alkyl. In some embodiments, each R^{b} is independently C₁-C₆ alkoxyl. In some embodiments, each R^{b} is independently C₁-C₆ haloalkyl. In some embodiments, each R^{b} is independently C₃-C₂₂ cycloalkyl. In some embodiments, each R^{b} is independently C₄-C₁₀ heterocycle. In some embodiments, each R^{b} is independently C₆-C₁₀ aryl. In some embodiments, each R^{b} is independently or C₅-C₉ heteroaryl.

In some embodiments, p is 0-10. In some embodiments, p is 1-10. In some embodiments, p is 2-10. In some embodiments, p is 3-10. In some embodiments, p is 4-10. In some embodiments, p is 5-10. In some embodiments, p is 6-10. In some embodiments, p is 7-10. In some embodiments, p is 8-10. In some embodiments, p is 9-10. In some embodiments, p is 10. In some embodiments, p is 9. In some embodiments, p is 8. In some embodiments, p is 7. In some embodiments, p is 6. In some embodiments, p is 5. In some embodiments, p is 4. In some embodiments, p is 3. In some embodiments, p is 2. In some embodiments, p is 1.

In some embodiments, the disclosure is directed to compounds of Formula VI.

In some embodiments, the disclosure is directed to pharmaceutically acceptable salts or solvates of compounds of Formula VI.

In some embodiments, provided are compounds having a formula of is

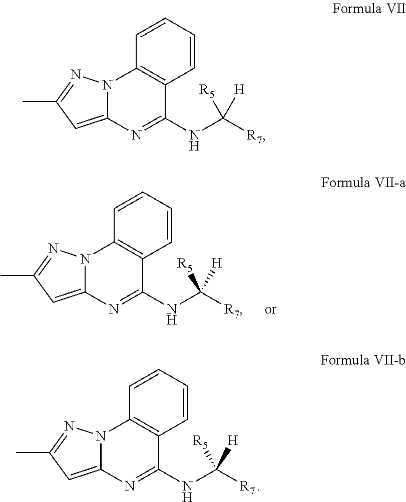

or pharmaceutically acceptable salts or solvates thereof, wherein the variables are as defined herein. In some embodiments, the compound has a formula of

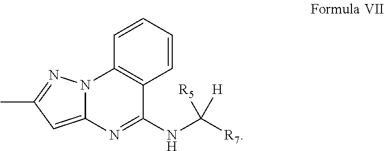

In some embodiments, the compound has a formula of

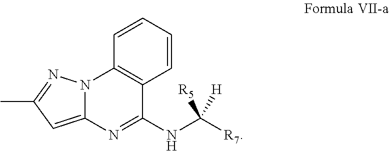

In some embodiments, the compound has a formula of

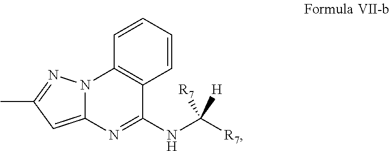

In some embodiments. In some embodiments, R₅ is H. In some embodiments, R₅ is CH₃. In some embodiments, R₅ is CO₂H. In some embodiments, R₅ is or CO₂Me. R₅ is H. In some embodiments, R₅ is CH₃. In some embodiments, R₅ is CO₂H. In some embodiments, R₅ is CO₂Me.

In some embodiments, the disclosure is directed to compounds of Formula VII.

In some embodiments, the disclosure is directed to compounds of Formula VII-a.

In some embodiments, the disclosure is directed to compounds of Formula VII-b.

In some embodiments, the disclosure is directed to pharmaceutically acceptable salts or solvates of compounds of Formula VII.

In some embodiments, the disclosure is directed to pharmaceutically acceptable salts or solvates of compounds of Formula VII-a.

In some embodiments, the disclosure is directed to pharmaceutically acceptable salts or solvates of compounds of Formula VII-b.

In some embodiments, R₇ is C₃-C₂₂ cycloalkyl. In some embodiments, R₇ is

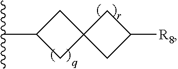

wherein the variables are as defined herein. In some embodiments, q is 0-8. In some embodiments, q is 1-8. In some embodiments, q is 2-8. In some embodiments, q is 3-8. In some embodiments, q is 4-8. In some embodiments, q is 5-8. In some embodiments, q is 6-8. In some embodiments, q is 7-8. In some embodiments, q is 8. In some embodiments, q is 7. In some embodiments, q is 6. In some embodiments, q is 5. In some embodiments, q is 4. In some embodiments, q is 3. In some embodiments, q is 2. In some embodiments, q is 1. In some embodiments, r is 0-8. In some embodiments, r is 1-8. In some embodiments, r is 2-8. In some embodiments, r is 3-8. In some embodiments, r is 4-8. In some embodiments, r is 5-8. In some embodiments, r is 6-8. In some embodiments, r is 7-8. In some embodiments, r is 8. In some embodiments, r is 7. In some embodiments, r is 6. In some embodiments, r is 5. In some embodiments, r is 4. In some embodiments, r is 3. In some embodiments, r is 2. In some embodiments, r is 1. In some embodiments, R₈ is H, D, halogen, R^{c}, —C(O)R^{c}, —CO₂R^{c}, —S(O)R^{c}, SO₂R^{c}, —S(O)NHR^{c}, —SO₂NHR^{c}. In some embodiments, R₈ is —C(O)NHR^{c}. In some embodiments, R₈ is —N(R^{c})CO₂R^{c}. In some embodiments, R₈ is or N(R^{c})CONR^{c}. In some embodiments. In some embodiments, R₈ is H. In some embodiments, R₈ is D. In some embodiments, R₈ is halogen. In some embodiments, R₈ is R^{c}. In some embodiments, R₈ is —C(O)R^{c}. In some embodiments, R₈ is —CO₂R^{c}. In some embodiments, R₈ is —S(O)R^{c}. In some embodiments, R₈ is SO₂R^{c}. In some embodiments, R₈ is —S(O)NHR^{c}. In some embodiments, R₈ is —SO₂NHR^{c}. In some embodiments, R₈ is —C(O)NHR^{c}. In some embodiments, Rx is —N(R^{c})CO₂R^{c}. In some embodiments, R₈ is N(R^{c})CONR^{c}. In some embodiments, each R₈ is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₆-C₁₀ heterocycle, C₆-C₁₀ aryl, or C₅-C₉ heteroaryl. In some embodiments, each R^{c} is independently H. In some embodiments, each R^{c} is independently D. In some embodiments, each R^{c} is independently CF₃. In some embodiments, each R^{c} is independently OCF₃. In some embodiments, each R^{c} is independently CN. In some embodiments, each R^{c} is independently SF₃. In some embodiments, each R^{c} is independently SF₅. In some embodiments, each R^{c} is independently halogen. In some embodiments, each R^{c} is independently optionally substituted C₁-C₆ alkyl. In some embodiments, each R^{c} is independently C₁-C₆ alkoxyl. In some embodiments, each R^{c} is independently C₁-C₆ haloalkyl. In some embodiments, each R^{c} is independently C₃-C₂₂ cycloalkyl. In some embodiments, each R^{c} is independently C₄-C₁₀ heterocycle. In some embodiments, each R^{c} is independently C₆-C₁₀ aryl. In some embodiments, each R^{c} is independently C₅-C₉ heteroaryl.

In some embodiments, R₇ is

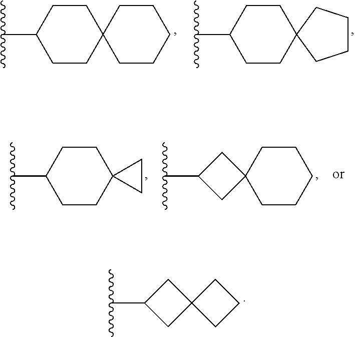

In some embodiments, R₇ is

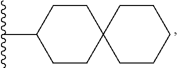 [SMILES: *C1CCC2(CCCCC2)CC1]

In some embodiments, R₇ is

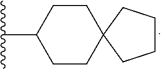 [SMILES: *C1CCC2(CCCC2)CC1]

In some embodiments, R₇ is

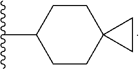 [SMILES: *C1CCC2(CC1)CC2]

In some embodiments, R₇ is

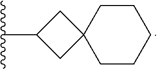 [SMILES: *C1CC2(CCCCC2)C1]

In some embodiments, R₇ is

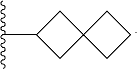 [SMILES: *C1CC2(CCC2)C1]

In some embodiments, R₇ is

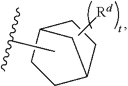

wherein the variables are as defined herein.

In some embodiments, R^{d} is H, D, halogen, R^{e}, —C(O)R^{e}, —CO₂R^{e}, —S(O)R^{e}, SO₂R^{e}, —S(O)NHR^{e}, —SO₂NHR^{e}, —C(O)NHR^{e}, —N(R^{e})CO₂R^{e}, or N(R^{e})CONR^{e}. In some embodiments, R^{d} is H. In some embodiments, R^{d} is D. In some embodiments, R^{d} is halogen. In some embodiments, R^{d} is R^{e}. In some embodiments, R^{d} is —C(O)R^{e}. In some embodiments, R^{d} is —CO₂R^{e}. In some embodiments, R^{d} is —S(O)R^{e}. In some embodiments, R^{d} is SO₂R^{e}. In some embodiments, R^{d} is —S(O)NHR^{e}. In some embodiments, R^{d} is —SO₂NHR^{e}. In some embodiments, R^{d} is —C(O)NHR^{e}. In some embodiments, R^{d} is —N(R^{e})CO₂R^{e}. In some embodiments, R^{d} is N(R^{e})CONR^{e}. In some embodiments, each R^{e} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl. In some embodiments, each R^{e} is independently H. In some embodiments, each R^{e} is independently D. In some embodiments, each R^{e} is independently CF₃. In some embodiments, each R^{e} is independently OCF₃. In some embodiments, each R^{e} is independently CN. In some embodiments, each R^{e} is independently SF₃. In some embodiments, each R^{e} is independently SF₅. In some embodiments, each R^{e} is independently halogen. In some embodiments, each R^{e} is independently optionally substituted C₁-C₆ alkyl. In some embodiments, each R^{e} is independently C₁-C₆ alkoxyl. In some embodiments, each R^{e} is independently C₁-C₆ haloalkyl. In some embodiments, each R^{e} is independently C₃-C₂ cycloalkyl. In some embodiments, each R^{e} is independently C₄-C₁₀ heterocycle. In some embodiments, each R^{e} is independently C₆-C₁₀ aryl. In some embodiments, each R^{e} is independently or C₅-C₉ heteroaryl.

In some embodiments, t is 0-12. In some embodiments, t is 1-12. In some embodiments, t is 2-12. In some embodiments, t is 3-12. In some embodiments, t is 4-12. In some embodiments, t is 5-12. In some embodiments, t is 6-12. In some embodiments, t is 7-12. In some embodiments, t is 8-12. In some embodiments, t is 9-12. In some embodiments, t is 10-12. In some embodiments, t is 11-12. In some embodiments, t is 10. In some embodiments, tis 11. In some embodiments, t is 12. In some embodiments, t is 9. In some embodiments, t is 8. In some embodiments, t is 7. In some embodiments, t is 6. In some embodiments, t is 5. In some embodiments, t is 4. In some embodiments, t is 3. In some embodiments, t is 2. In some embodiments, t is 1.

In some embodiments, R₇ is

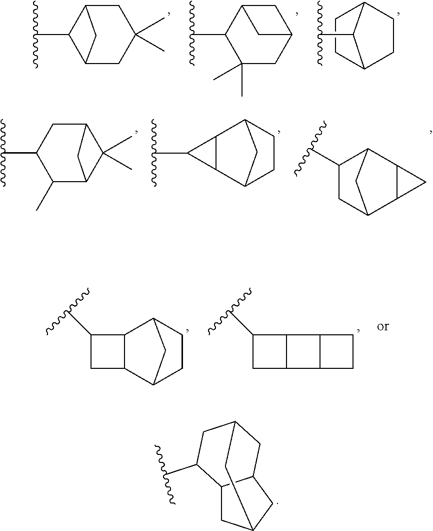

In some embodiments, R₇ is

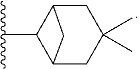 [SMILES: *C1C2CC1CC(C)(C)C2]

In some embodiments, R₇ is

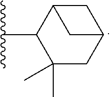 [SMILES: *C1C2CC(C2)CC1(C)C]

In some embodiments, R₇ is

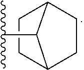 [SMILES: *C1C2CCC1CC2]

In some embodiments, R₇ is

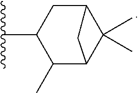 [SMILES: *C1CC2CC(C1C)C2(C)C]

In some embodiments, R₇ is

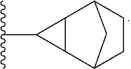 [SMILES: *C1C2C3CCC(C3)C12]

In some embodiments, R₇ is

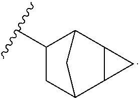 [SMILES: *C1CC2CC1C1CC21]

In some embodiments, R₇ is

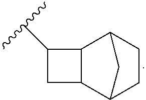 [SMILES: *C1CC2C3CCC(C3)C12]

In some embodiments, R₇ is

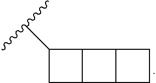 [SMILES: *C1CC2C3CCC3C12]

In some embodiments, R₇ is

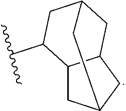 [SMILES: *C1CC2CC3CC(C2)C1C3]

In some embodiments, R₇ is

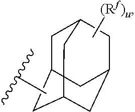

wherein the variables are as defined herein. In some embodiments, R^{f} is H, D, halogen, R^{g}, —C(O)R^{g}, —CO₂R^{g}, —S(O)R^{g}, SO₂R^{g}, —S(O)NHR^{g}, —SO₂NHR^{g}, —C(O)NHR^{g}, —N(R^{g})CO₂R^{g}, or N(R^{g})CONR^{g}. In some embodiments, R^{f} is H. In some embodiments, R^{f} is D. In some embodiments, R^{f} is halogen. In some embodiments, R^{f} is R^{g}. In some embodiments, R^{f} is —C(O)R^{g}. In some embodiments, R^{f} is —CO₂R^{g}. In some embodiments, R_{f} is —S(O)R^{g}. In some embodiments, R_{f} is SO₂R^{g}. In some embodiments, R^{f} is —S(O)NHR^{g}. In some embodiments, R^{f} is —SO₂NHR⁸. In some embodiments, R^{f} is —C(O)NHR^{g}. In some embodiments, R^{f} is —N(R^{g})CO₂R^{g}. In some embodiments, R_{f} is or N(R^{g})CONR^{g}. In some embodiments, each R^{g} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl. In some embodiments, each R^{g} is independently H. In some embodiments, each R^{g} is independently D. In some embodiments, each R^{g} is independently CF₃. In some embodiments, each R^{g} is independently OCF₃. In some embodiments, each R^{g} is independently CN. In some embodiments, each R^{g} is independently SF₃. In some embodiments, each R^{g} is independently SF₅. In some embodiments, each R^{g} is independently halogen. In some embodiments, each R^{g} is independently optionally substituted C₁-C₆ alkyl. In some embodiments, each R^{g} is independently C₁-C₆ alkoxyl. In some embodiments, each R^{g} is independently C₁-C₆ haloalkyl. In some embodiments, each R^{g} is independently C₃-C₂₂ cycloalkyl. In some embodiments, each R^{g} is independently C₄-C₁₀ heterocycle. In some embodiments, each R^{g} is independently C₆-C₁₀ aryl. In some embodiments, each R^{g} is independently or C₅-C₉ heteroaryl. In some embodiments, u is 0-12. In some embodiments, u is 1-12. In some embodiments, u is 2-12. In some embodiments, u is 3-12. In some embodiments, u is 4-12. In some embodiments, u is 5-12. In some embodiments, u is 6-12. In some embodiments, u is 7-12. In some embodiments, u is 8-12. In some embodiments, u is 9-12. In some embodiments, u is 10-12. In some embodiments, u is 11-12. In some embodiments, u is 10. In some embodiments, u is 11. In some embodiments, u is 12. In some embodiments, u is 9. In some embodiments, u is 8. In some embodiments, u is 7. In some embodiments, u is 6. In some embodiments, u is 5. In some embodiments, u is 4. In some embodiments, u is 3. In some embodiments, u is 2. In some embodiments, u is 1.

In some embodiments, R₇ is

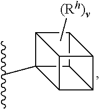

wherein the variables are as defined herein. In some embodiments, R^{h} is H, D, halogen, R^{j}, —C(O)R^{j}, —CO₂R^{j}, —S(O)R^{j}, SO₂R^{j}, —S(O)NHR^{j}, —SO₂NHR^{j}, —C(O)NHR^{j}, —N(R^{j})CO₂R^{j}, or N(R^{j})CONR^{j}. In some embodiments, R^{h} is H. In some embodiments, R^{h} is D. In some embodiments, R^{h} is halogen. In some embodiments, R^{h} is R^{j}. In some embodiments, R^{h} is —C(O)R^{j}. In some embodiments, R^{h} is —CO₂R^{j}. In some embodiments, R^{h} is —S(O)R^{j}. In some embodiments, R^{h} is SO₂R^{j}. In some embodiments, R^{h} is —S(O)NHR^{j}. In some embodiments, R^{h} is —SO₂NHR^{j}. In some embodiments, R^{h} is —C(O)NHR^{j}. In some embodiments, R^{h} is —N(R^{j})CO₂R^{j}. In some embodiments, R^{h} is N(R^{j})CONR^{j}. In some embodiments, each R^{j} is independently H. In some embodiments, each R^{j} is independently D. In some embodiments, each R^{j} is independently CF₃. In some embodiments, each R^{j} is independently OCF₃. In some embodiments, each R^{j} is independently CN. In some embodiments, each R^{j} is independently SF₃. In some embodiments, each Riis independently SF₅. In some embodiments, each R^{j} is independently halogen. In some embodiments, each R^{j} is independently optionally substituted C₁-C₆ alkyl. In some embodiments, each R^{j} is independently C₁-C₆ alkoxyl. In some embodiments, each R^{j} is independently C₁-C₆ haloalkyl. In some embodiments, each R^{j} is independently C₃-C₂₂ cycloalkyl. In some embodiments, each R^{j} is independently C₄-C₁₀ heterocycle. In some embodiments, each R^{j} is independently C₆-C₁₀ aryl. In some embodiments, each R^{j} is independently C₅-C₉ heteroaryl. In some embodiments, v is 0-7. In some embodiments, v is 1-7. In some embodiments, v is 2-7. In some embodiments, v is 3-7. In some embodiments, v is 4-7. In some embodiments, v is 5-7. In some embodiments, v is 6-7. In some embodiments, v is 7. In some embodiments, v is 6. In some embodiments, v is 5. In some embodiments, v is 4. In some embodiments, v is 3. In some embodiments, v is 2. In some embodiments, v is 1.

In some embodiments, R₇ is

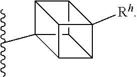

In some embodiments, R^{h} is H, F, CN, NH₂, COOH, SO₂Cl, CH₂NH₂, CH₂OH, CH₂COOH, C≡CH, CH₂COOMe. In some embodiments, R^{h} is H. In some embodiments, R^{h} is F. In some embodiments, R^{h} is CN. In some embodiments, R^{h} is NH₂. In some embodiments, R^{h} is COOH. In some embodiments, R^{h} is SO₂Cl. In some embodiments, R^{h} is CH₂NH₂. In some embodiments, R^{h} is CH₂OH. In some embodiments, R^{h} is CH₂COOH. In some embodiments, R^{h} is C≡CH. In some embodiments, R^{h} is CH₂COOMe,

In some embodiments, R₇ is

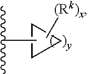

wherein the variables are as defined herein. In some embodiments, R^{k} is H, D, halogen, R^{m}, —C(O)R^{m}, —CO₂R^{m}, —S(O)R^{m}, SO₂R^{m}, —S(O)NHR^{m}, —SO₂NHR^{m}, —C(O)NHR^{m}, —N(R^{m})CO₂R^{m}, or N(R^{m})CONR^{m}. In some embodiments, R^{k} is H, D, halogen, R^{m}, —C(O)R^{m}, —CO₂R^{m}, —S(O)R^{m}, SO₂R^{m}, —S(O)NHR^{m}, —SO₂NHR^{m}, —C(O)NHR^{m}, —N(R^{m})CO₂R^{m}, or N(R^{m})CONR^{m}. In some embodiments, R^{k} is H. In some embodiments, R^{k} is D. In some embodiments, R^{k} is halogen. In some embodiments, R^{k} is R^{m}. In some embodiments, R^{k} is —C(O)R^{m}. In some embodiments, R^{k} is —CO₂R^{m}. In some embodiments, R^{k} is —S(O)R^{m}. In some embodiments, R^{k} is SO₂R^{m}. In some embodiments, R^{k} is —S(O)NHR^{m}. In some embodiments, R^{k} is —SO₂NHR^{m}. In some embodiments, R^{k} is —C(O)NHR^{m}. In some embodiments, R^{k} is —N(R^{m})CO₂R^{m}. In some embodiments, R^{k} is or N(R^{m})CONR^{m}. In some embodiments. In some embodiments, R^{k} is R^{k} is H. In some embodiments, R^{k} is D. In some embodiments, R^{k} is halogen. In some embodiments, R^{k} is R^{m}. In some embodiments, R^{k} is —C(O)R^{m}. In some embodiments, R^{k} is —CO₂R^{m}. In some embodiments, R^{k} is —S(O)R^{m}. In some embodiments, R^{k} is SO₂R^{m}. In some embodiments, R^{k} is —S(O)NHR^{m}. In some embodiments, R^{k} is —SO₂NHR^{m}. In some embodiments, R^{k} is —C(O)NHR^{m}. In some embodiments, R^{k} is —N(R^{m})CO₂R^{m}. In some embodiments, R^{k} is or N(R^{m})CONR^{m}. In some embodiments, each R^{m} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl. In some embodiments, each R^{m} is independently H. In some embodiments, each R^{m} is independently D. In some embodiments, each R^{m} is independently CF₃. In some embodiments, each R^{m} is independently OCF₃. In some embodiments, each R^{m} is independently CN. In some embodiments, each R^{m} is independently SF₃. In some embodiments, each R^{m} is independently SF₃. In some embodiments, each R^{m} is independently halogen. In some embodiments, each R^{m} is independently optionally substituted C₁-C₆ alkyl. In some embodiments, each R^{m} is independently C₁-C₆ alkoxyl. In some embodiments, each R^{m} is independently C₁-C₆ haloalkyl. In some embodiments, each R^{m} is independently C₃-C₂₂ cycloalkyl. In some embodiments, each R^{m} is independently C₄-C₁₀ heterocycle. In some embodiments, each R^{m} is independently C₆-C₁₀ aryl. In some embodiments, each R^{m} is independently or C₅-C₉ heteroaryl. In some embodiments, y is 1-12. In some embodiments, y is 2-12. In some embodiments, y is 3-12. In some embodiments, y is 4-12. In some embodiments, y is 5-12. In some embodiments, y is 6-12. In some embodiments, y is 7-12. In some embodiments, y is 8-12. In some embodiments, y is 9-12. In some embodiments, y is 10-12. In some embodiments, y is 11-12. In some embodiments, y is 10. In some embodiments, y is 11. In some embodiments, y is 12. In some embodiments, y is 9. In some embodiments, y is 8. In some embodiments, y is 7. In some embodiments, y is 6. In some embodiments, y is 5. In some embodiments, y is 4. In some embodiments, y is 3. In some embodiments, y is 2. In some embodiments, y is 1. In some embodiments, x is 0-12. In some embodiments, x is 1-12. In some embodiments, x is 2-12. In some embodiments, x is 3-12. In some embodiments, x is 4-12. In some embodiments, x is 5-12. In some embodiments, x is 6-12. In some embodiments, x is 7-12. In some embodiments, x is 8-12. In some embodiments, x is 9-12. In some embodiments, x is 10-12. In some embodiments, x is 11-12. In some embodiments, x is 10. In some embodiments, x is 11. In some embodiments, x is 12. In some embodiments, x is 9. In some embodiments, x is 8. In some embodiments, x is 7. In some embodiments, x is 6. In some embodiments, x is 5. In some embodiments, x is 4. In some embodiments, x is 3. In some embodiments, x is 2. In some embodiments, x is 1.

In some embodiments, R^{k} is F.

In some embodiments, R^{k} is Me.

In some embodiments, the compound has a formula of

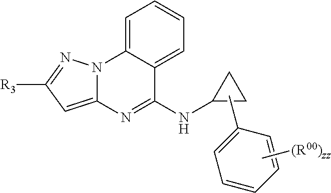

Formula VIII, wherein R₃ and y are defined above, R⁰⁰ is C₁-C₆ alkyl, C₁-C₆ alkenyl, C₁-C₆ alkynyl, C₁-C₆ alkoxy, phenyl, C₃-C₅ heteroaryl, C₃-C₁₀ cycloalkyl, C₅-C₆ aryloxy, —CN, —OH, oxo, halo, haloalkyl, —NO₂, —CO₂H, —NH₂, —NH(C₁-C₈alkyl), —N(C₁-C₈alkyl)₂, —NH(phenyl), —N(phenyl)₂, —CHO, —CO(C₁-C₆ alkyl), —CO(phenyl), —CO₂(C₁-C₆ alkyl), or —CO₂(phenyl), and zz is 0-5. In some embodiments, R₃ is H or optionally substituted C₁-C₆ alkyl. In some embodiments, y is 0-6. In some embodiments, R⁰⁰ is halo or haloalkyl. In some embodiments, zz is 0-2.

In some embodiments, R⁷ is

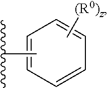

wherein the variables are as defined herein. In some embodiments, R^{o} is H, D, halogen, R^{p}, —C(O)R^{p}, —CO₂R^{p}, —S(O)R^{p}, SO₂R^{p}, —S(O)NHR^{p}, —SO₂NHR^{p}, —C(O)NHR^{p}, —N(R^{p})CO₂R^{p}, or N(R^{p})CONR^{p}. In some embodiments, R^{o} is H. In some embodiments, R^{o} is D. In some embodiments, R^{o} is halogen. In some embodiments, R^{o} is R^{p}. In some embodiments, R^{o} is —C(O)R^{p}. In some embodiments, R^{o} is —CO₂R^{p}. In some embodiments, R^{o} is —S(O)R^{p}. In some embodiments, R^{o} is SO₂R^{p}. In some embodiments, R^{o} is —S(O)NHR^{p}. In some embodiments, R^{o} is —SO₂NHR^{p}. In some embodiments, R^{o} is —C(O)NHR^{p}. In some embodiments, R^{o} is —N(R^{p})CO₂R^{p}. In some embodiments, R^{o} is N(R^{p})CONR^{p}. In some embodiments, each R^{p} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl. In some embodiments, each R^{p} is independently H. In some embodiments, each R^{p} is independently D. In some embodiments, each R^{p} is independently CF₃. In some embodiments, each R^{p} is independently OCF₃. In some embodiments, each R^{p} is independently CN. In some embodiments, each R^{p} is independently SF₃. In some embodiments, each R^{p} is independently SF₅. In some embodiments, each R^{p} is independently halogen. In some embodiments, each R^{p} is independently optionally substituted C₁-C₆ alkyl. In some embodiments, each R^{p} is independently C₁-C₆ alkoxyl. In some embodiments, each R^{p} is independently C₁-C₆ haloalkyl. In some embodiments, each R^{p} is independently C₃-C₂₂ cycloalkyl. In some embodiments, each R^{p} is independently C₄-C₁₀ heterocycle. In some embodiments, each R^{p} is independently C₆-C₁₀ aryl. In some embodiments, each R^{p} is independently or C₅-C₉ heteroaryl.

In some embodiments, z is 0-5. In some embodiments, z is 0-4. In some embodiments, z is 1-4. In some embodiments, z is 2-4. In some embodiments, z is 0-4. In some embodiments, z is 3-4. In some embodiments, z is 0. In some embodiments, z is 1. In some embodiments, z is 2. In some embodiments, z is 3. In some embodiments, z is 4. In some embodiments, z is 5.

In some embodiments, R^{o} is H, D, CF₃, OCF₃, CN, or NR^{q}₂. In some embodiments, each R^{q} is independently H. In some embodiments, each R^{q} is independently D. In some embodiments, each R^{q} is independently CF₃. In some embodiments, each R^{q} is independently OCF₃. In some embodiments, each R^{q} is independently CN. In some embodiments, each R^{q} is independently SF₃. In some embodiments, each R^{q} is independently SF₅. In some embodiments, each R^{q} is independently halogen. In some embodiments, each R^{q} is independently optionally substituted C₁-C₆ alkyl. In some embodiments, each R^{q} is independently C₁-C₆ alkoxyl. In some embodiments, each R^{q} is independently C₁-C₆ haloalkyl. In some embodiments, each R^{q} is independently C₃-C₂₂ cycloalkyl. In some embodiments, each R^{q} is independently C₄-C₁₀ heterocycle. In some embodiments, each R^{q} is independently C₆-C₁₀ aryl. In some embodiments, each R^{q} is independently or C₅-C₉ heteroaryl. In some embodiments, two R^{q} together form a C₄-C₁₀ heterocycle.

In some embodiments, R₇ is optionally substituted C₁-C₆ alkyl. In some embodiments, R₇ is

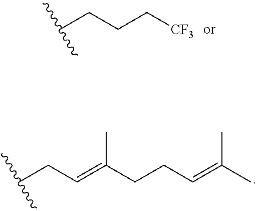 [SMILES: *CCCC(F)(F)F ; *C/C=C(\C)CCC=C(C)C]

In some embodiments, R₇ is

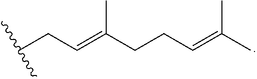 [SMILES: *C/C=C(\C)CCC=C(C)C]

In some embodiments, R₇ is

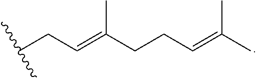 [SMILES: *C/C=C(\C)CCC=C(C)C]

In some embodiments, R₇ is

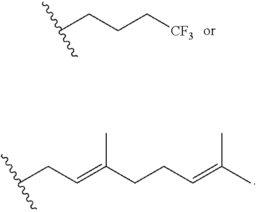 [SMILES: *CCCC(F)(F)F ; *C/C=C(\C)CCC=C(C)C]

In some embodiments, the compound described and provided for herein has a formula of

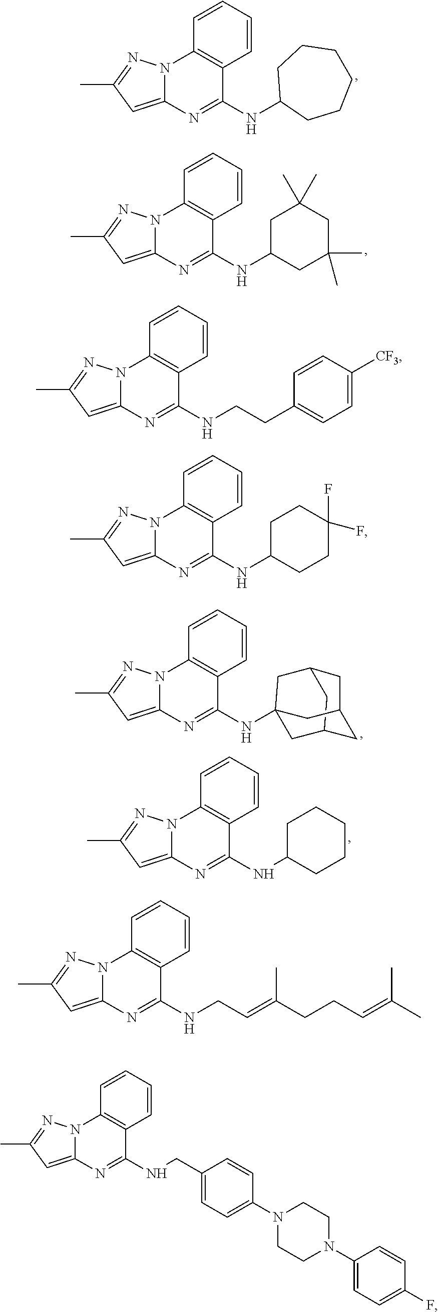

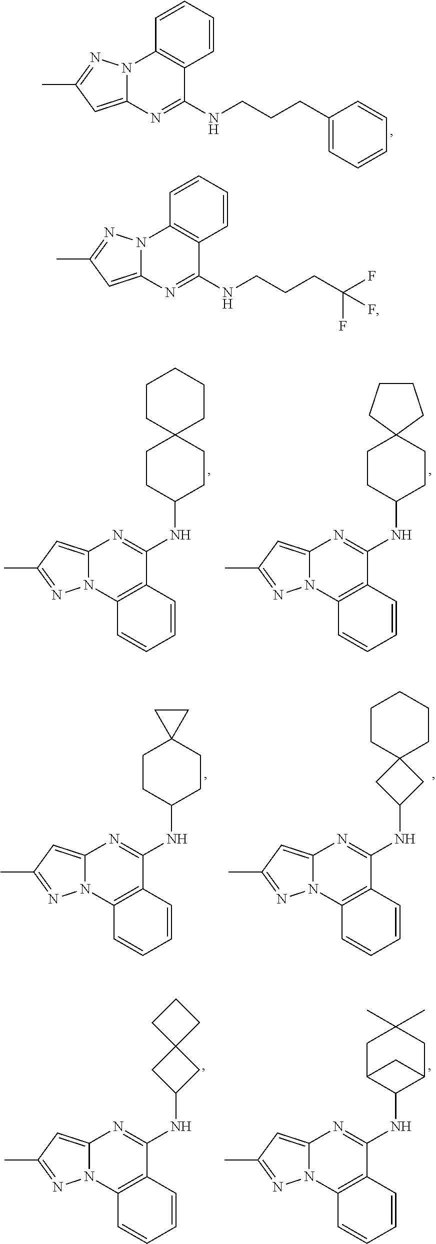

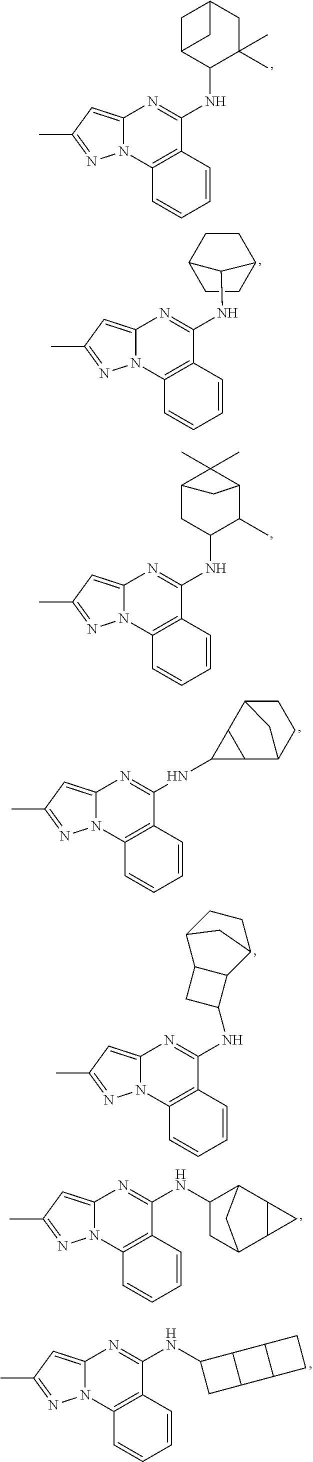

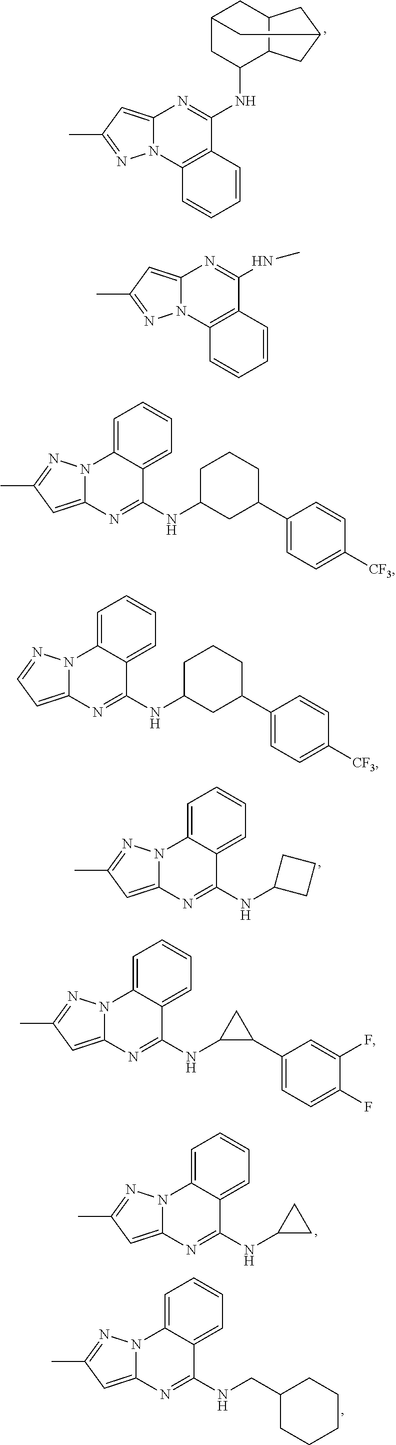

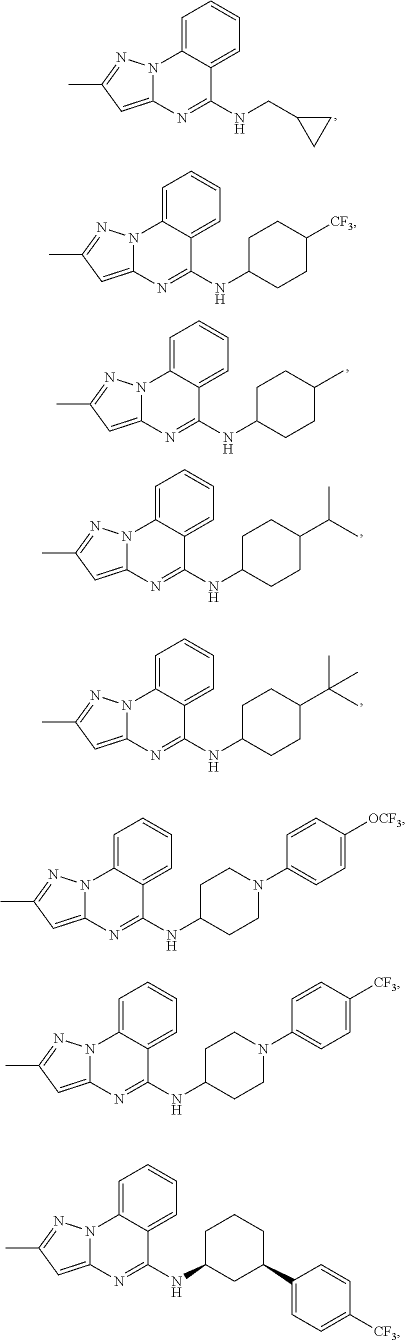

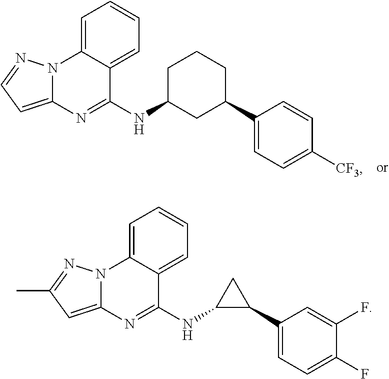 [SMILES: FC(F)(F)c1ccc([C@@H]2CCC[C@H](Nc3nc4ccnn4c4ccccc34)C2)cc1 ; Cc1cc2nc(N[C@@H]3C[C@H]3c3ccc(F)c(F)c3)c3ccccc3n2n1]

In some embodiments, the compound has a formula of

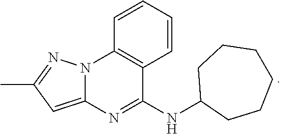 [SMILES: Cc1cc2nc(NC3CCCCCC3)c3ccccc3n2n1]

In some embodiments, the compound has a formula of

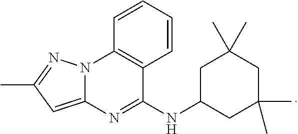

In some embodiments, the compound has a formula of

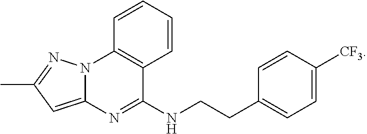 [SMILES: Cc1cc2nc(NCCc3ccc(C(F)(F)F)cc3)c3ccccc3n2n1]

In some embodiments, the compound has a formula of

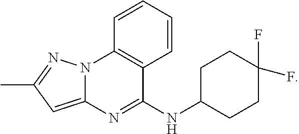 [SMILES: Cc1cc2nc(NC3CCC(F)(F)CC3)c3ccccc3n2n1]

In some embodiments, the compound has a formula of

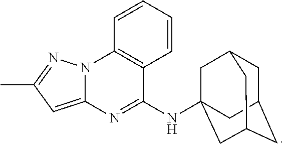 [SMILES: Cc1cc2nc(NC34CC5CC(CC(C5)C3)C4)c3ccccc3n2n1]

In some embodiments, the compound has a formula of

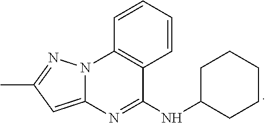 [SMILES: Cc1cc2nc(NC3CCCCC3)c3ccccc3n2n1]

In some embodiments, the compound has a formula of

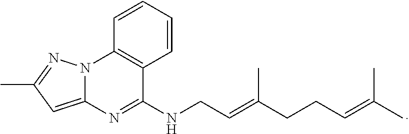 [SMILES: CC(C)=CCC/C(C)=C/CNc1nc2cc(C)nn2c2ccccc12]

In some embodiments, the compound has a formula of

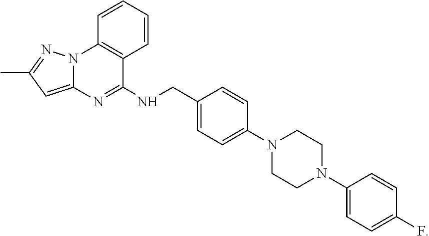 [SMILES: Cc1cc2nc(NCc3ccc(N4CCN(c5ccc(F)cc5)CC4)cc3)c3ccccc3n2n1]

In some embodiments, the compound has a formula of

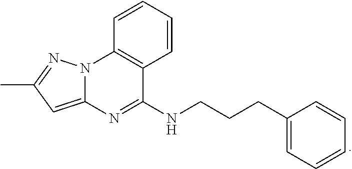 [SMILES: Cc1cc2nc(NCCCc3ccccc3)c3ccccc3n2n1]

In some embodiments, the compound has a formula of

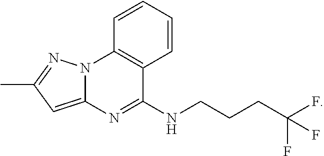 [SMILES: Cc1cc2nc(NCCCC(F)(F)F)c3ccccc3n2n1]

In some embodiments, the compound has a formula of

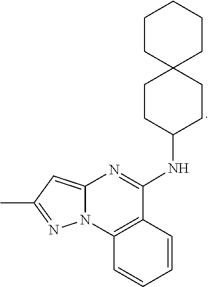 [SMILES: Cc1cc2nc(NC3CCC4(CCCCC4)CC3)c3ccccc3n2n1]

In some embodiments, the compound has a formula of

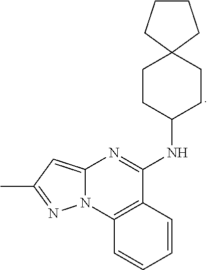 [SMILES: Cc1cc2nc(NC3CCC4(CCCC4)CC3)c3ccccc3n2n1]

In some embodiments, the compound has a formula of

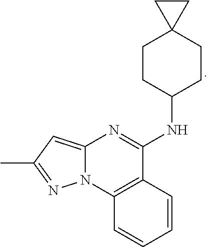 [SMILES: Cc1cc2nc(NC3CCC4(CC3)CC4)c3ccccc3n2n1]

In some embodiments, the compound has a formula of

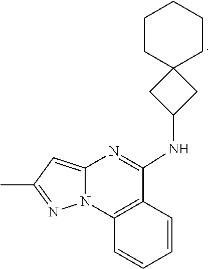 [SMILES: Cc1cc2nc(NC3CC4(CCCCC4)C3)c3ccccc3n2n1]

In some embodiments, the compound has a formula of

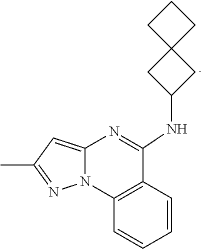 [SMILES: Cc1cc2nc(NC3CC4(CCC4)C3)c3ccccc3n2n1]

In some embodiments, the compound has a formula of

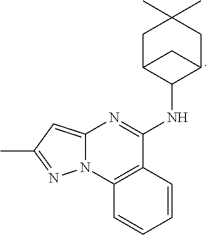

In some embodiments, the compound has a formula of

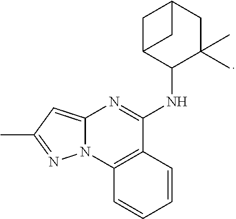

In some embodiments, the compound has a formula of

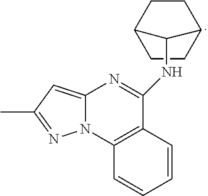 [SMILES: Cc1cc2nc(NC3C4CCC3CC4)c3ccccc3n2n1]

In some embodiments, the compound has a formula of

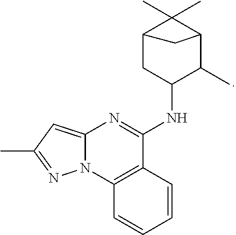

In some embodiments, the compound has a formula of

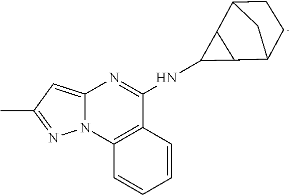 [SMILES: Cc1cc2nc(NC3C4C5CCC(C5)C34)c3ccccc3n2n1]

In some embodiments, the compound has a formula of

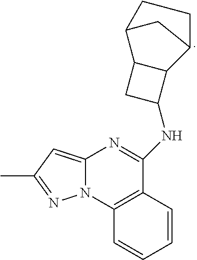 [SMILES: Cc1cc2nc(NC3CC4C5CCC(C5)C34)c3ccccc3n2n1]

In some embodiments, the compound has a formula of

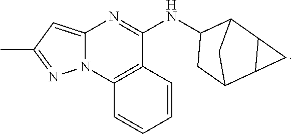 [SMILES: Cc1cc2nc(NC3CC4CC3C3CC43)c3ccccc3n2n1]

In some embodiments, the compound has a formula of

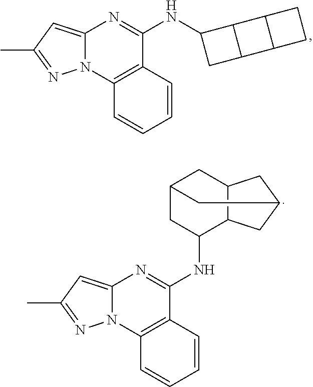 [SMILES: Cc1cc2nc(NC3CC4C5CCC5C34)c3ccccc3n2n1 ; Cc1cc2nc(NC3CC4CCC5CC(C4)C3C5)c3ccccc3n2n1]

In some embodiments, the compound has a formula of

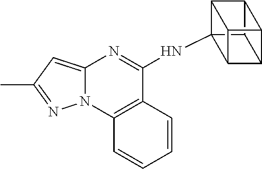 [SMILES: Cc1cc2nc(NC34C5C6C7C5C3C7C64)c3ccccc3n2n1]

In some embodiments, the compound has a formula of

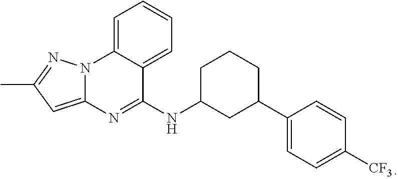 [SMILES: Cc1cc2nc(NC3CCCC(c4ccc(C(F)(F)F)cc4)C3)c3ccccc3n2n1]

In some embodiments, the compound has a formula of

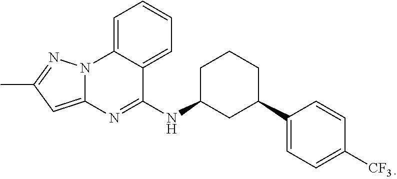 [SMILES: Cc1cc2nc(N[C@H]3CCC[C@@H](c4ccc(C(F)(F)F)cc4)C3)c3ccccc3n2n1]

In some embodiments, the compound has a formula of

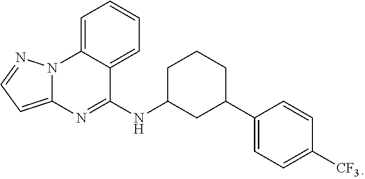 [SMILES: FC(F)(F)c1ccc(C2CCCC(Nc3nc4ccnn4c4ccccc34)C2)cc1]

In some embodiments, the compound has a formula of

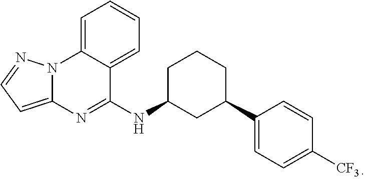 [SMILES: FC(F)(F)c1ccc([C@@H]2CCC[C@H](Nc3nc4ccnn4c4ccccc34)C2)cc1]

In some embodiments, the compound has a formula of

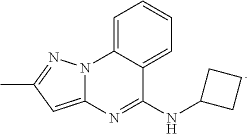 [SMILES: Cc1cc2nc(NC3CCC3)c3ccccc3n2n1]

In some embodiments, the compound has a formula of

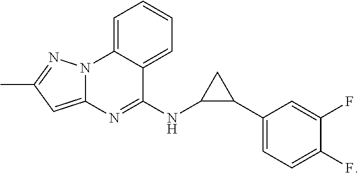 [SMILES: Cc1cc2nc(NC3CC3c3ccc(F)c(F)c3)c3ccccc3n2n1]

In some embodiments, the compound has a formula of

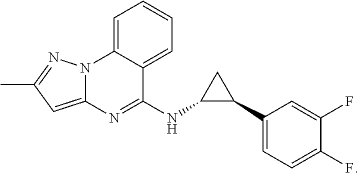 [SMILES: Cc1cc2nc(N[C@@H]3C[C@H]3c3ccc(F)c(F)c3)c3ccccc3n2n1]

In some embodiments, the compound has a formula of

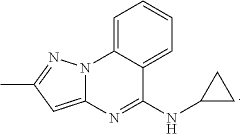 [SMILES: Cc1cc2nc(NC3CC3)c3ccccc3n2n1]

In some embodiments, the compound has a formula of

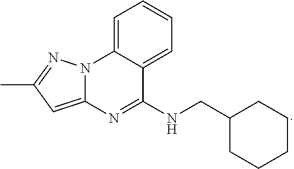 [SMILES: Cc1cc2nc(NCC3CCCCC3)c3ccccc3n2n1]

In some embodiments, the compound has a formula of

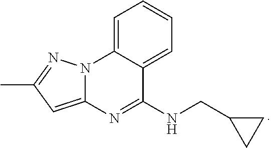 [SMILES: Cc1cc2nc(NCC3CC3)c3ccccc3n2n1]

In some embodiments, the compound has a formula of

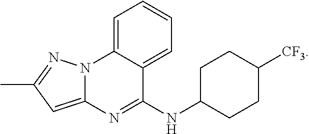 [SMILES: Cc1cc2nc(NC3CCC(C(F)(F)F)CC3)c3ccccc3n2n1]

In some embodiments, the compound has a formula of

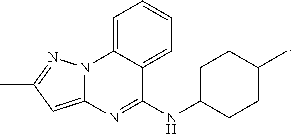

In some embodiments, the compound has a formula of

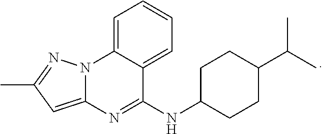 [SMILES: Cc1cc2nc(NC3CCC(C(C)C)CC3)c3ccccc3n2n1]

In some embodiments, the compound has a formula of

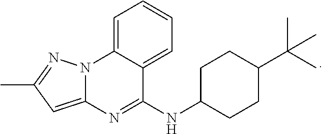 [SMILES: Cc1cc2nc(NC3CCC(C(C)(C)C)CC3)c3ccccc3n2n1]

In some embodiments, the compound has a formula of

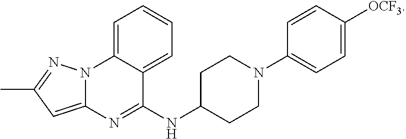 [SMILES: Cc1cc2nc(NC3CCN(c4ccc(OC(F)(F)F)cc4)CC3)c3ccccc3n2n1]

In some embodiments, the compound has a formula of

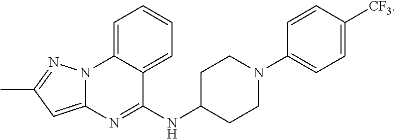 [SMILES: Cc1cc2nc(NC3CCN(c4ccc(C(F)(F)F)cc4)CC3)c3ccccc3n2n1]

Exemplary compounds of the disclosure are set forth in Table 1, below. Table 1: Exemplary Compounds

| Compound | IUPAC | Structure |
|---|---|---|
| 1 | 2-methyl-N-(4- (trifluoromethyl)phenethyl) pyrazolo[1,5-a]quinazolin- 5-amine | 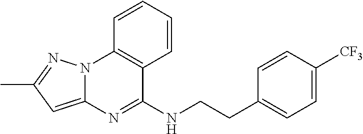 [SMILES: Cc1cc2nc(NCCc3ccc(C(F)(F)F)cc3)c3ccccc3n2n1] |
| 2 | 2-methyl-N-(3,3,5,5- tetramethylcyclohexyl) pyrazolo[1,5-a] quinazolin-5-amine | 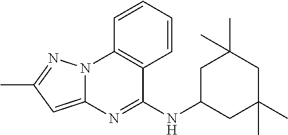 |
| 3 | N-(4,4-difluorocyclohexyl)-2- methylpyrazolo[1,5-a] quinazolin-5-amine | 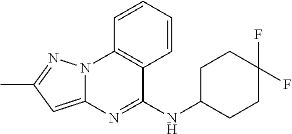 [SMILES: Cc1cc2nc(NC3CCC(F)(F)CC3)c3ccccc3n2n1] |
| 4 | N-((3s,5s,7s)-adamantan-1-yl)- 2-methylpyrazolo[1,5-a] quinazolin-5-amine | 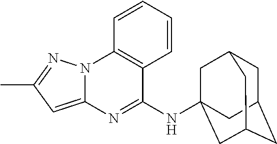 [SMILES: Cc1cc2nc(NC34CC5CC(CC(C5)C3)C4)c3ccccc3n2n1] |
| 5 | N-cyclohexyl-2- methylpyrazolo[1,5-a] quinazolin-5-amine | 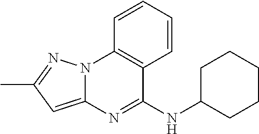 [SMILES: Cc1cc2nc(NC3CCCCC3)c3ccccc3n2n1] |
| 6 | N-(4-(4- (4-fluorophenyl)piperazin- 1-yl)benzyl)-2- methylpyrazolo[1,5-a] quinazolin-5-amine | 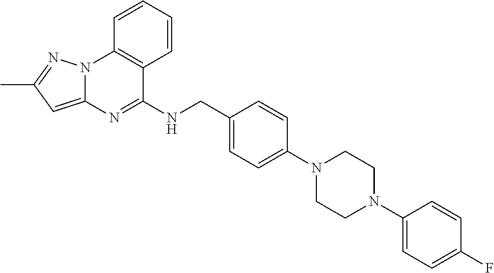 [SMILES: Cc1cc2nc(NCc3ccc(N4CCN(c5ccc(F)cc5)CC4)cc3)c3ccccc3n2n1] |
| 7 | S-chloro-2- methylpyrazolo[1,5-a] quinazoline | 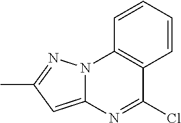 |
| 8 | N-cyclopentyl-2- methylpyrazolo[1,5-a] quinazolin-5-amine | 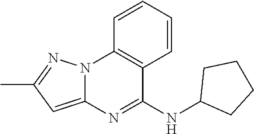 [SMILES: Cc1cc2nc(NC3CCCC3)c3ccccc3n2n1] |
| 9 | N-cyclooctyl-2- methylpyrazolo[1,5-a] quinazolin-5-amine | 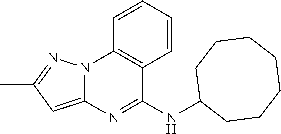 [SMILES: Cc1cc2nc(NC3CCCCCCC3)c3ccccc3n2n1] |
| 10 | 2-methyl-N-(4,4,4- trifluorobutyl)pyrazolo[1,5-a] quinazolin-5-amine | 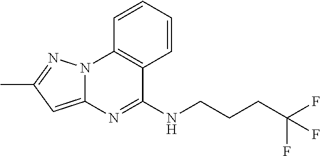 [SMILES: Cc1cc2nc(NCCCC(F)(F)F)c3ccccc3n2n1] |
| 11 | 2-methyl-N-(3- phenylpropyl)pyrazolo[1,5-a] quinazolin-5-amine | 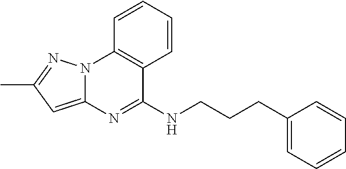 [SMILES: Cc1cc2nc(NCCCc3ccccc3)c3ccccc3n2n1] |
| 12 | N-cycloheptyl-2- methylpyrazolo[1,5-a] quinazolin-5-amine | 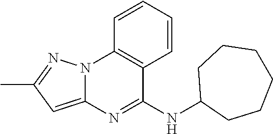 [SMILES: Cc1cc2nc(NC3CCCCCC3)c3ccccc3n2n1] |
| 13 | (E)-N-(3,7-dimethylocta- 2,6-dien-1-yl)- 2-methylpyrazolo[1,5-a] quinazolin-5-amine | 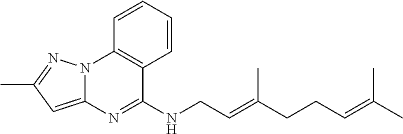 [SMILES: CC(C)=CCC/C(C)=C/CNc1nc2cc(C)nn2c2ccccc12] |
| 14 | 2-methyl-N-(4-(4- (trifluoromethyl)phenoxy) phenyl)pyrazolo[1,5-a] quinazolin-5-amine | 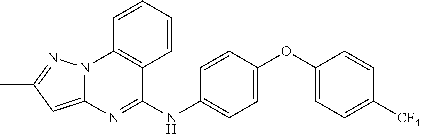 |
| 15 | 2-methyl-N-(3-(4- (trifluoromethyl)phenyl) cyclohexy1)pyrazolo [1,5-a]quinazolin-5-amine | 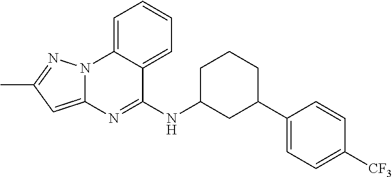 [SMILES: Cc1cc2nc(NC3CCCC(c4ccc(C(F)(F)F)cc4)C3)c3ccccc3n2n1] |
| 15A | 2-methyl-N-((1S,3R)-3-(4- (trifluoromethyl)phenyl) cyclohexyl)pyrazolo[1,5-a] quinazolin-5-amine | 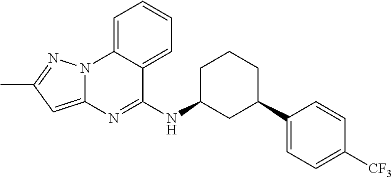 [SMILES: Cc1cc2nc(N[C@H]3CCC[C@@H](c4ccc(C(F)(F)F)cc4)C3)c3ccccc3n2n1] |
| 16 | N-(3-(4- (trifluoromethyl)phenyl) cyclohexyl)pyrazolo[1,5-a] quinazolin-5-amine | 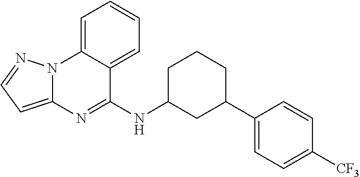 [SMILES: FC(F)(F)c1ccc(C2CCCC(Nc3nc4ccnn4c4ccccc34)C2)cc1] |
| 16A | N-((1S,3R)-3-(4- (trifluoromethyl)phenyl) cyclohexyl)pyrazolo[1,5-a] quinazolin-5-amine | 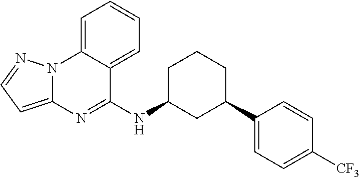 [SMILES: FC(F)(F)c1ccc([C@@H]2CCC[C@H](Nc3nc4ccnn4c4ccccc34)C2)cc1] |
| 17 | 2-methyl-N-(spiro[3.3]heptan-2- yl)pyrazolo[1,5-a] quinazolin-5-amine | 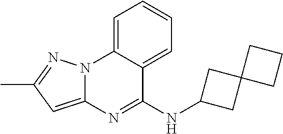 [SMILES: Cc1cc2nc(NC3CC4(CCC4)C3)c3ccccc3n2n1] |
| 18 | N-cyclobutyl-2- methylpyrazolo[1,5-a] quinazolin-5-amine | 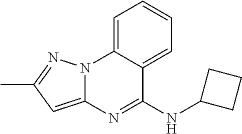 [SMILES: Cc1cc2nc(NC3CCC3)c3ccccc3n2n1] |
| 19 | N-(2-(3,4- difluorophenyl)cyclopropyl)- 2-methylpyrazolo[1,5-a] quinazolin-5-amine | 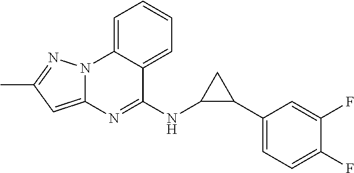 [SMILES: Cc1cc2nc(NC3CC3c3ccc(F)c(F)c3)c3ccccc3n2n1] |
| 19A | N-((1R,2S)-2-(3,4- difluorophenyl)cyclopropyl)- 2-methylpyrazolo[1,5-a] quinazolin-5-amine | 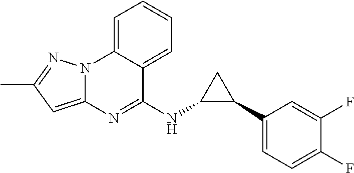 [SMILES: Cc1cc2nc(N[C@@H]3C[C@H]3c3ccc(F)c(F)c3)c3ccccc3n2n1] |
| 20 | N-cyclopropyl-2- methylpyrazolo[1,5-a] quinazolin-5-amine | 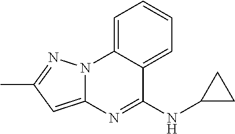 [SMILES: Cc1cc2nc(NC3CC3)c3ccccc3n2n1] |
| 21 | N-(cyclohexylmethyl)-2- methylpyrazolo[1,5-a] quinazolin-5-amine | 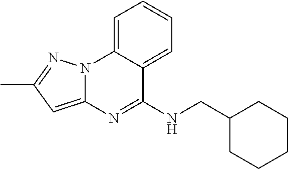 [SMILES: Cc1cc2nc(NCC3CCCCC3)c3ccccc3n2n1] |
| 22 | N-(cyclopropylmethyl)-2- methylpyrazolo[1,5-a] quinazolin-5-amine | 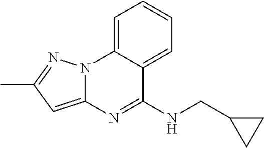 [SMILES: Cc1cc2nc(NCC3CC3)c3ccccc3n2n1] |
| 23 | 2-methyl-N-(4- (trifluoromethyl)cyclohexyl) pyrazolo[1,5-a] quinazolin-5-amine | 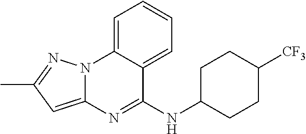 [SMILES: Cc1cc2nc(NC3CCC(C(F)(F)F)CC3)c3ccccc3n2n1] |
| 24 | 2-methyl-N-(4- methylcyclohexyl)pyrazolo [1,5-a]quinazolin- 5-amine | 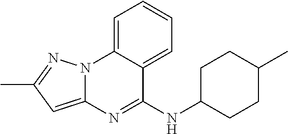 |
| 25 | N-(4-isopropylcyclohexy])-2- methylpyrazolo[1,5-a] quinazolin-5-amine | 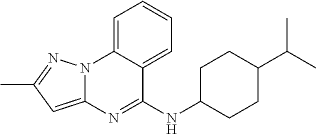 [SMILES: Cc1cc2nc(NC3CCC(C(C)C)CC3)c3ccccc3n2n1] |
| 26 | N-(4-(tert-butyl)cyclohexyl)-2- methylpyrazolo[1,5-a] quinazolin-5-amine | 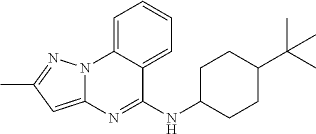 [SMILES: Cc1cc2nc(NC3CCC(C(C)(C)C)CC3)c3ccccc3n2n1] |
| 27 | 2-methyl-N-(1-(4- (trifluoromethoxy)phenyl) piperidin-4-yl)pyrazolo[1,5-a] quinazolin-5-amine | 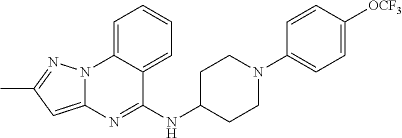 [SMILES: Cc1cc2nc(NC3CCN(c4ccc(OC(F)(F)F)cc4)CC3)c3ccccc3n2n1] |
| 28 | 2-methyl-N-(1-(4- (trifluoromethyl)phenyl) piperidin-4-yl)pyrazolo[1,5-a] quinazolin-5-amine | 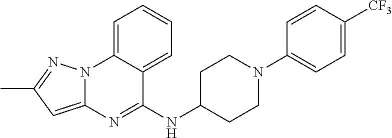 [SMILES: Cc1cc2nc(NC3CCN(c4ccc(C(F)(F)F)cc4)CC3)c3ccccc3n2n1] |

### Uses, Formulation and Administration:

### Pharmaceutically Acceptable Compositions

In some embodiments, provided are pharmaceutical compositions comprising a compound, or a pharmaceutically acceptable salt thereof, as described herein. According to another embodiment, the present disclosure provides a composition comprising a compound of the present disclosure or a pharmaceutically acceptable derivative thereof and a pharmaceutically acceptable carrier, adjuvant, or vehicle. In some embodiments, provided are pharmaceutical compositions comprising a compound, or a pharmaceutically acceptable salt thereof, as described herein and a pharmaceutically acceptable carrier, adjuvant, or vehicle. In some embodiments, provided are pharmaceutical compositions comprising a compound, or a pharmaceutically acceptable salt thereof, as described herein and a pharmaceutically acceptable carrier. In some embodiments, provided are pharmaceutical compositions comprising a compound, or a pharmaceutically acceptable salt thereof, as described herein and a pharmaceutically acceptable adjuvant. In some embodiments, provided are pharmaceutical compositions comprising a compound, or a pharmaceutically acceptable salt thereof, as described herein and a pharmaceutically acceptable vehicle

The amount of compound in compositions of the present disclosure is such that is effective to measurably inhibit Cytochrome BD oxidase, or a mutant thereof, in a biological sample or in a patient. In certain embodiments, the amount of compound in compositions of the present disclosure is such that is effective to measurably inhibit Cytochrome BD oxidase, or a mutant thereof, in a biological sample or in a patient. In certain embodiments, a composition of the present disclosure is formulated for administration to a patient in need of such composition. In some embodiments, a composition of the present disclosure is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of the present disclosure that, upon administration to a recipient, is capable of providing, either directly or indirectly, a compound of the present disclosure or an inhibitorily active metabolite or residue thereof.

As used herein, the term “inhibitorily active metabolite or residue thereof” means that a metabolite or residue thereof is also an inhibitor of Cytochrome BD oxidase, or a mutant thereof.

Compositions of the present disclosure may be administered orally, parenterally, by inhalation spray, topically, rectally, nasally, buccally, vaginally, or via an implanted reservoir. The term “parenteral” as used herein includes subcutaneous, intravenous, intramuscular, intra-articular, intra-synovial, intrasternal, intrathecal, intrahepatic, intralesional, and intracranial injection or infusion techniques. Preferably, the compositions are administered orally, intraperitoneally, or intravenously. Sterile injectable forms of the compositions of the present disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms, may also be used for the purposes of formulation.

Pharmaceutically acceptable compositions of the present disclosure may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of the present disclosure may be administered in the form of suppositories for rectal administration. These can be prepared by mixing the agent with a suitable non-irritating excipient that is solid at room temperature but liquid at rectal temperature and, therefore, will melt in the rectum to release the drug. Such materials include cocoa butter, beeswax, and polyethylene glycols.

Pharmaceutically acceptable compositions of the present disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of the present disclosure include, but are not limited to, mineral oil, liquid petrolatum, white petrolatum, propylene glycol, polyoxyethylene, polyoxypropylene compound, emulsifying wax, and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water. In some embodiments, compounds may be applied in pure form for topical administration, e.g., when they are liquids. However, it will generally be desirable to administer the active agent to the skin as a composition or formulation, for example, in combination with a dermatologically acceptable carrier, which may be a solid, a liquid, a gel, or the like. For example, suitable solid carriers include finely divided solids such as talc, clay, microcrystalline cellulose, silica, alumina, and the like. Suitable liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer. Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of the present disclosure are formulated for oral administration. Such formulations may be administered with or without food. In some embodiments, pharmaceutically acceptable compositions of the present disclosure are administered without food. In other embodiments, pharmaceutically acceptable compositions of the present disclosure are administered with food.

The amount of compounds of the present disclosure that may be combined with the carrier materials to produce a composition in a single dosage form will vary depending upon the host treated and the particular mode of administration. Preferably, provided compositions should be formulated so that a dosage of between 0.01-100 mg/kg body weight/day of the inhibitor can be administered to a patient receiving these compositions. In some embodiments, a suitable dose will be in the range of from about 0.5 to about 100 mg/kg, e.g., from about 10 to about 75 mg/kg of body weight per day, such as 3 to about 50 mg per kilogram bodyweight of the recipient per day, preferably in the range of 6 to 90 mg/kg/day, most preferably in the range of 15 to 60 mg/kg/day. The compound is conveniently formulated in a unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound described herein can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four, or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations; such as multiple inhalations from an insufflator or by application of a plurality of drops into the eye.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

### Uses of Compounds and Pharmaceutically Acceptable Compositions

Compounds and compositions described herein are generally useful for the inhibition of Cytochrome BD oxidase, or a mutant thereof.

The activity of a compound utilized in the present disclosure as an inhibitor of Cytochrome BD oxidase, or a mutant thereof, may be assayed in vitro, in vivo or in a cell line. In vitro assays include assays that determine inhibition of Cytochrome BD oxidase, or a mutant thereof. Alternate in vitro assays quantitate the ability of the inhibitor to bind to Cytochrome BD oxidase, or a mutant thereof. Detailed conditions for assaying a compound utilized in the present disclosure as an inhibitor of Cytochrome BD oxidase, or a mutant thereof, are set forth in the Examples below.

As used herein, the terms “treatment,” “treat,” or “treating” of any disease or disorder refers, in some embodiments, to ameliorating the disease or disorder (i.e., arresting or reducing the development of the disease or at least one of the clinical symptoms thereof). In another embodiment, “treating,” “treat,” or “treatment” refers to ameliorating at least one physical parameter, which may not be discernible by the subject. In yet another embodiment, “treatment,” “treat,” or “treating” refers to modulating the disease or disorder, either physically (e.g., stabilization of a discernible symptom), physiologically (e.g, stabilization of a physical parameter), or both. In yet another embodiment, “treatment,” “treat,” or “treating” refers to delaying the onset of the disease or disorder.

Provided compounds are inhibitors of Cytochrome BD oxidase, or a mutant thereof, and are therefore useful for treating a variety of diseases, disorders, or conditions associated with regulating the activity of Cytochrome BD oxidase. Accordingly, in certain embodiments, the present disclosure provides a method for treating a low hepcidin disorder, disease, and/or condition, comprising the step of administering to a patient in need thereof a compound of the present disclosure or pharmaceutically acceptable composition thereof.

In some embodiments, the present disclosure provides methods for treating infections in a mammal, which involve administering to a mammal having an infection an effective amount of a compound or a pharmaceutical composition described herein. A mammal includes a primate, human, rodent, canine, feline, bovine, ovine, equine, swine, caprine, bovine, and the like.

Without wishing to be bound by any specific theory, inhibition of Cytochrome BD oxidase has been found to lead to the death of mycobacterial. Accordingly, in some embodiments, the present disclosure provides methods for a mycobacterial infection in a subject, comprising administering to the subject one or more compounds as described and provided for herein, or a pharmaceutically acceptable salt thereof.

In some embodiments, the present disclosure provides methods for treating a mycobacterial infection in a subject, comprising administering to the subject one or more compounds as described and provided for herein. In some embodiments, the present disclosure provides methods for treating a mycobacterial infection in a subject, comprising administering to the subject a pharmaceutically acceptable salt of a compound as described and provided for herein. In some embodiments, the present disclosure provides methods for treating a mycobacterial infection in a subject, comprising administering to the subject pharmaceutically acceptable salts of two or more compounds as described and provided for herein.

In some embodiments, the present disclosure provides methods for treating a mycobacterial infection in a subject, comprising administering to the subject a pharmaceutical composition comprising one or more compounds as described and provided for herein, or a pharmaceutically acceptable salt thereof. In some embodiments, the present disclosure provides methods for treating a mycobacterial infection in a subject, comprising administering to the subject a pharmaceutical composition comprising one or more compounds as described and provided for herein. In some embodiments, the present disclosure provides methods for treating a mycobacterial infection in a subject, comprising administering to the subject a pharmaceutical composition comprising a compound as described and provided for herein. In some embodiments, the present disclosure provides methods for treating a mycobacterial infection in a subject, comprising administering to the subject a pharmaceutical composition comprising two or more compounds as described and provided for herein.

In some embodiments, the present disclosure provides methods for treating a mycobacterial infection in a subject, comprising administering to the subject a pharmaceutical composition comprising a pharmaceutically acceptable salt of a compound as described and provided for herein. In some embodiments, the present disclosure provides methods for treating a mycobacterial infection in a subject, comprising administering to the subject a pharmaceutical composition comprising pharmaceutically acceptable salts of two or more compounds as described and provided for herein.

In some embodiments, the present disclosure provides methods for treating a mycobacterial infection in a subject, comprising administering to the subject a compound has a formula of

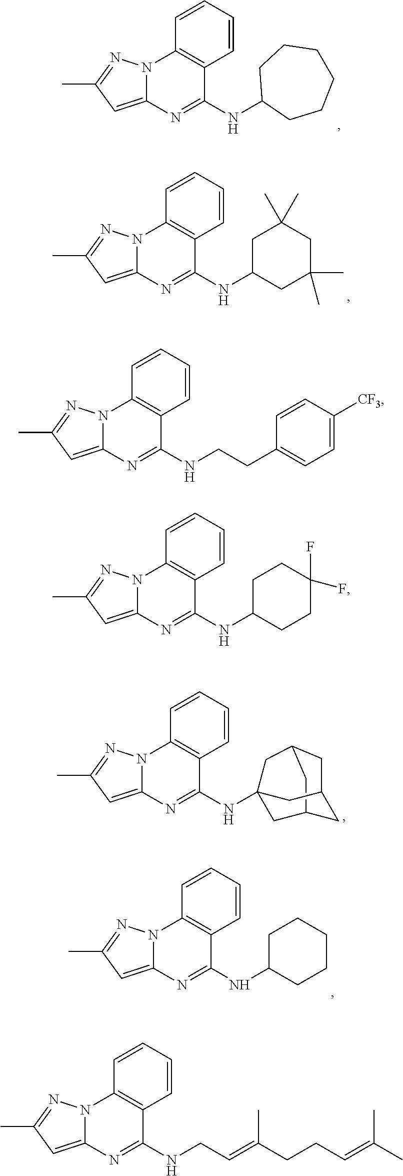

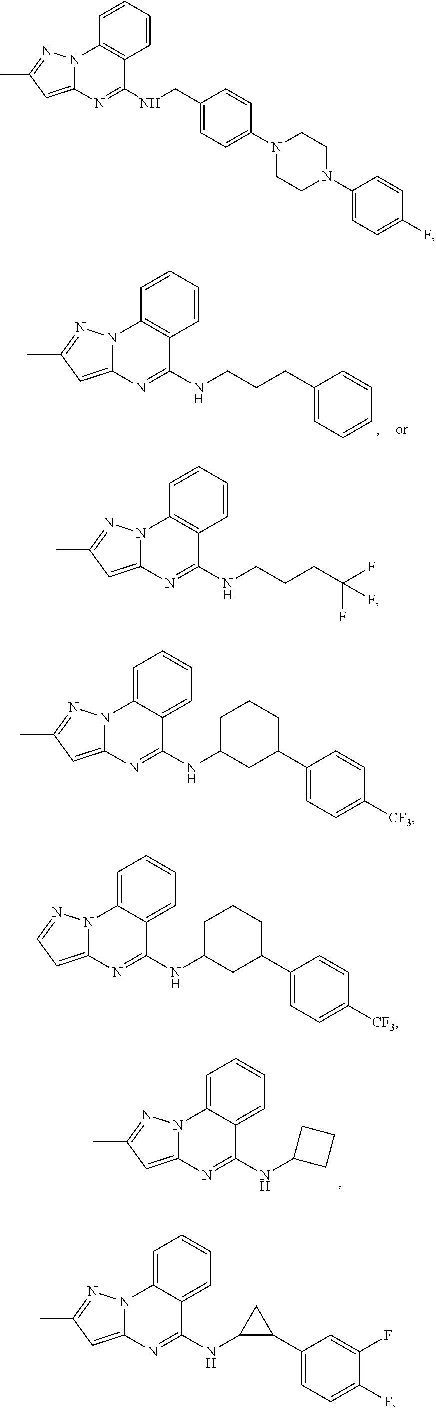

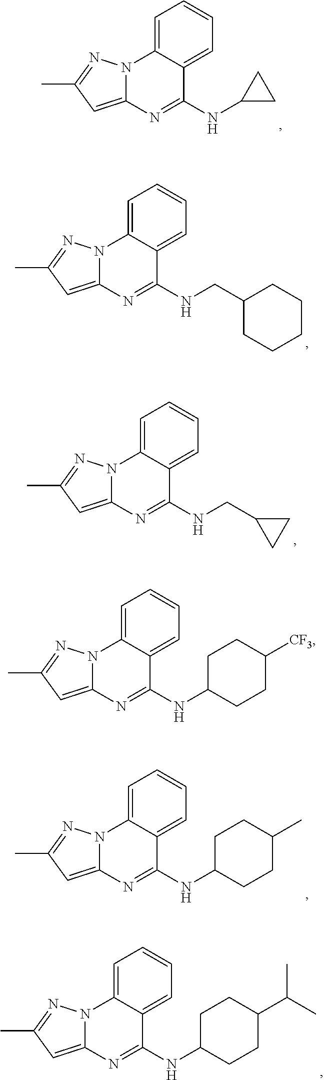

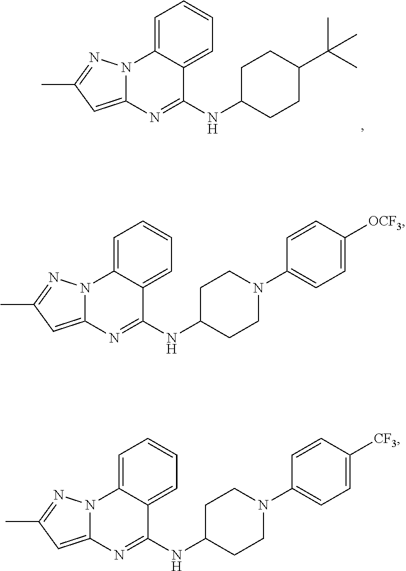

or a pharmaceutically acceptable salt thereof, or a pharmaceutical composition thereof. In some embodiments, the methods for treating a mycobacterial infection in a subject, comprising administering to the subject a compound has a formula of

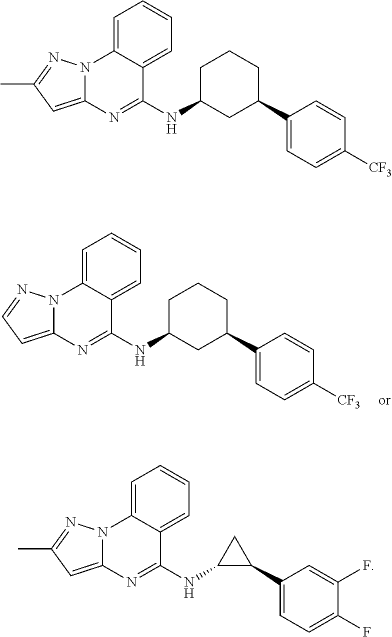 [SMILES: Cc1cc2nc(N[C@H]3CCC[C@@H](c4ccc(C(F)(F)F)cc4)C3)c3ccccc3n2n1 ; FC(F)(F)c1ccc([C@@H]2CCC[C@H](Nc3nc4ccnn4c4ccccc34)C2)cc1 ; Cc1cc2nc(N[C@@H]3C[C@H]3c3ccc(F)c(F)c3)c3ccccc3n2n1]

In some embodiments, the present disclosure provides methods for treating a mycobacterial infection as described herein, comprising administering to the subject a compound as described and provided for herein, or a pharmaceutically acceptable salt thereof, or a pharmaceutical composition thereof, wherein the compound is present in a therapeutically effective amount.

In some embodiments, the present disclosure provides methods for treating a mycobacterial infection as described herein, further comprising administering an inhibitor of the oxidative phosphorylation processes in mycobacteria. In some embodiments, the inhibitor of the oxidative phosphorylation processes in mycobacteria is a QcrB inhibitor. In some embodiments, the QcrB inhibitor is in the imidazopyridine class. In some embodiments, the QcrB inhibitor is Q203 or clofazimine or the like. In some embodiments, there is a synergistic effect between the compound as described herein and the inhibitor as described herein. In some embodiments, the compound or the pharmaceutically acceptable salt thereof as described herein is administered in a synergistic therapeutically effective amount. In some embodiments, the inhibitor or the pharmaceutically acceptable salt thereof as described herein is administered in a synergistic therapeutically effective amount.

In some embodiments, the present disclosure provides methods for treating a mycobacterial infection in a subject, comprising administering to the subject the pharmaceutical composition as described herein. In some embodiments, the compound or the pharmaceutically acceptable salt thereof, is present in a therapeutically effective amount. In some embodiments, there is a synergistic effect between the compound as described herein and the inhibitor as described herein. In some embodiments, the compound or the pharmaceutically acceptable salt thereof as described herein is administered in a synergistic therapeutically effective amount. In some embodiments, the inhibitor or the pharmaceutically acceptable salt thereof as described herein is administered in a synergistic therapeutically effective amount.

In some embodiments, the present disclosure provides methods for treating a mycobacterial infection in a subject as described and provided for herein, wherein the mycobacterial infection is caused by a bacteria from the Mycobacterium tuberculosis complex.

In some embodiments, the present disclosure provides methods for treating a mycobacterial infection in a subject as described and provided for herein, wherein the mycobacterial infection is caused by a non-tuberculosis mycobacteria (NTM) such as those belonging to the Mycobacterium abscessus complex or to the Mycobacterium avium complex.

In some embodiments, the present disclosure provides methods for treating a mycobacterial infection in a subject as described and provided for herein, wherein the subject is in need thereof.

Although the present embodiments have been described in connection with certain specific embodiments for instructional purposes, the present embodiments are not limited thereto. Accordingly, various modifications, adaptations, and combinations of various features of the described embodiments can be practiced without departing from the scope of the presently claimed subject matter as set forth in the claims. Furthermore, the following examples are illustrative, but not limiting, of the compounds, compositions, and methods described herein. Other suitable modifications and adaptations known to those skilled in the art are within the scope of the following embodiments. Any and all journal articles, patent applications, issued patents, or other cited references are incorporated by reference in their entirety.

### EXAMPLES

### Synthesis

Compounds of the present disclosure, including salts thereof, can be prepared using known organic synthesis techniques and can be synthesized according to any of the numerous possible synthetic routes.

The reactions for preparing compounds of the present disclosure can be carried out in suitable solvents, which can be readily selected by one of skill in the art of organic synthesis. Suitable solvents can be substantially nonreactive with the starting materials (reactants), the intermediates, or products at the temperatures at which the reactions are carried out, e.g., temperatures, which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H NMR or ¹³C NMR), infrared spectroscopy, spectrophotometry (e.g., UV-visible), or mass spectrometry, or by chromatography such as high-performance liquid chromatography (“HPLC”) or thin-layer chromatography, or liquid chromatography-mass spectrometry (“LC-MS”).

Unless otherwise stated, work-up includes distribution of the reaction mixture between the organic and aqueous phase indicated within parentheses, separation of layers and drying the organic layer over anhydrous sodium sulphate, filtration, and distillation of the solvent under reduced pressure. Purification, unless otherwise mentioned, includes purification by silica gel chromatographic techniques, generally using ethyl acetate/petroleum ether mixture of a suitable polarity as the mobile phase.

Compounds of the present disclosure can be prepared using numerous preparatory reactions known in the literature. The Schemes below provide general guidance in connection with preparing the compounds provided herein. One skilled in the art would understand that the preparations shown in the Schemes can be modified or optimized using general knowledge of organic chemistry to prepare various compounds. Example synthetic methods for preparing compounds are provided in the schemes below.

### General Schemes:

The general synthetic methods used in each General Schemes follow and include an illustration of a compound that was synthesized using the designated General Scheme. None of the specific conditions and reagents noted herein are to be construed as limiting the scope of the present disclosure and are provided for illustrative purposes only.

The following abbreviations refer respectively to the definitions below:

The expressions, “ambient temperature,” “room temperature,” and “r.t.” as used herein, are understood in the art, and generally refer to a temperature, e.g., a reaction temperature, that is about the temperature of the room in which the reaction is carried out, for example, a temperature from about 20° C. to about 30° C.

ACN—Acetonitrile; br—Broad; ° C.—Degree Celsius; CHCl₃—Chloroform; CD₃OD—Deuterated Methanol; DMSO—de-Deuterated dimethylsulfoxide; DCM—Dichloromethane; DIPEA—Diisopropylethylamine; DMF—N,N-Dimethylformamide; d—Doublet; dd—Doublet of doublet; EDC·HCl—1-(3-Dimethylaminopropyl)-3-ethylcarbodiimide hydrochloride, mg—Miligram; g—Gram; h—Hours; ¹H—Proton; HCl—Hydrochloric acid; HPLC—High-Performance Liquid Chromatography; H₂—Hydrogen, HOBt—1-Hydroxy benzotriazole, K₂CO₃—Potassium carbonate; LCMS—Liquid chromatography-mass spectroscopy; LiOH·H₂O—Lithium hydroxide monohydrate; M—Molar; MHz—Mega hertz (frequency); MeOH—Methanol, mL—MilliLiter; min—Minutes; mol—Moles; M⁺—Molecular ion; M—Multiplet; N₂—Nitrogen; NH₃—Ammonia; NBS—N-Bromosuccinimide; NCS—N-Chlorosuccinimide; NMR—Nuclear Magnetic Resonance; NaOH—Sodium Hydroxide; RT—Room temperature; s—Singlet; t—Triplet; TLC—Thin Layer Chromatography; TFA—Trifluoroacetic acid; TEA—Triethylamine; THF—Tetrahydrofuran; %—Percentage; μ—Micron; and δ—Delta; Zn—Zinc; mmol—millimoles.

Analysis for the compounds of the present disclosure, unless mentioned, was conducted in the general methods well known to the person skilled in the art. Having described the present disclosure with reference to certain preferred embodiments, other embodiments will become apparent to one skilled in the art from consideration of the specification. The present disclosure is further defined by reference to the following examples, describing in detail the analysis of the compounds of the present disclosure.

It will be apparent to those skilled in the art that many modifications, both to materials and methods, may be practiced without departing from the scope of the present disclosure.

### Materials and Methods

Unless otherwise stated, all reagents were obtained from commercial sources and were used as received without further purification. The NMR spectrometers utilized were Bruker instruments operating at frequencies at 400 MHz.

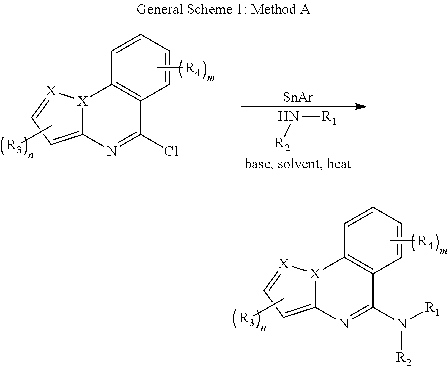

As shown in General scheme 1, the compounds as described herein can be prepared from classical Nucleophilic Aromatic Substitution (SNAr) reaction of the corresponding halogenated core (for example, 5-chloro-2-methylpyrazolo[1,5-a]quinazoline, CAS: 885525-06-4) and desired amine with an appropriate non-nucleophilic base (triethylamine, N,N-diisopropylethylamine, potassium carbonate) and non-nucleophilic solvent (such as acetonitrile, N,N-dimethylformamide, N,N-dimethylacetamide, dimethyl sulfoxide). The variables are as defined and provided for herein.

### Example 1: Synthesis of N-cycloheptyl-2-methylpyrazolo[1,5-a]quinazolin-5-amine (Compound 12) with Method A

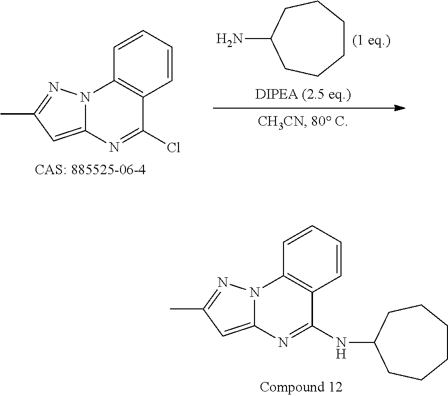 [SMILES: Cc1cc2nc(Cl)c3ccccc3n2n1 ; NC1CCCCCC1 ; Cc1cc2nc(NC3CCCCCC3)c3ccccc3n2n1]

5-Chloro-2-methylpyrazolo[1,5-a]quinazoline (CAS: 885525-06-4, 80 mg, 0.37 mmol), cycloheptanamine (CAS: 5452-35-7, 42 mg, 0.37 mmol) and N,N-diisopropylethylamine (0.16 mL, 0.92 mmol) were combined in 5 mL of acetonitrile. The reaction was heated at 80° C. for 24 h. Next, the reaction was cooled and concentrated in vacuo. The resulting residue was taken up in CH₂Cl₂, washed with 2% acetic acid aqueous solution (2×), water, and brine. The organic layer was dried over Na₂SO₄, filtered and organic solvent was evaporated. The resulting residue was purified by silica gel flash chromatography eluting with CH₂Cl₂: ethyl acetate to give N-cycloheptyl-2-methylpyrazolo[1,5-a]quinazolin-5-amine (Compound 12). ¹H NMR (400 MHZ, CDCl3) δ 8.30 (1H, dd, J=8.4, 0.7 Hz), 7.72 (1H, ddd, J=8.4, 7.3, 1.2 Hz), 7.62 (1H, dd, J=8.1, 0.6 Hz), 7.34 (1H, ddd, J=8.4, 7.3, 1.2 Hz), 6.03 (1H, s), 5.29 (1H, bd, J=7.5 Hz), 4.41 (1H, dquin, J=7.7, 3.8 Hz), 2.45 (3H, s), 2.21-2.06 (2H, m), 1.75-1.58 (10H, m). ¹³C NMR (125 MHz, CDCl₃) δ 152.5, 150.9, 146.9, 136.7, 132.7, 123.4, 122.1, 115.3, 111.2, 94.5, 51.3, 34.9, 28.3, 24.1, 14.6 Hz. HRMS (ESI-TOF, positive mode) m/z ([M+1]); Anal. Calcd for C₁₈H₂₂N₄ 295.192; Found 295.196.

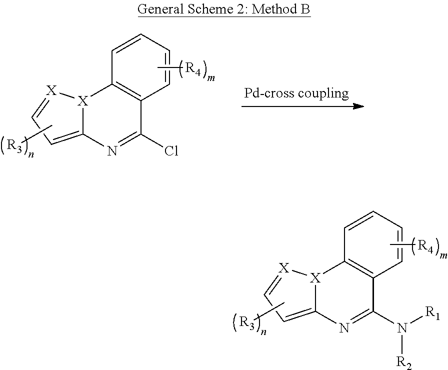

The compounds as described herein can be prepared by Palladium cross-coupling reactions, namely Buchwald-Hartwig amination, as shown in General scheme 2.

### Example 2: Synthesis of N-cycloheptyl-2-methylpyrazolo[1,5-a]quinazolin-5-amine (Compound 12) with Method B

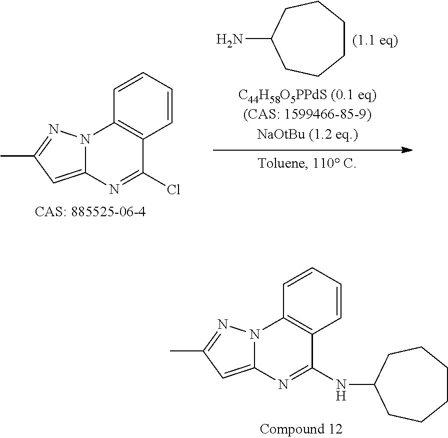 [SMILES: Cc1cc2nc(Cl)c3ccccc3n2n1 ; NC1CCCCCC1 ; Cc1cc2nc(NC3CCCCCC3)c3ccccc3n2n1]

In a vial, methanesulfonato-2′-methylamino-1,1′-biphenyl-2-yl-palladacycles generation 4 (CAS: 1599466-85-9, 15 mg, 0.017 mmol), sodium tert-butoxide (34 mg, 0.36 mmol), 5-chloro-2-methylpyrazolo[1,5-a]quinazoline (CAS: 885525-06-4, 42 mg, 0.19 mmol) and cycloheptanamine (CAS: 5452-35-7, 22 mg, 0197 mmol) were combined in anhydrous toluene (2 mL) to form a suspension. The suspension was stirred with argon degassing for 5 min. The reaction vial was sealed and heated to 110° C. for 48 h. Whereupon, the reaction was cooled, filtered through Celite to form a pad and the pad washed with CH₂Cl₂ (20 mL). The filtrate was washed with 2% acetic acid aqueous solution (2×), water, and brine. The organic layer was dried over Na₂SO₄ and filtered and the filtered organics were concentrated to dryness. The resulting residue was purified by silica gel flash chromatography to give N-cycloheptyl-2-methylpyrazolo[1,5-a]quinazolin-5-amine (Compound 12).

### Example 3: Synthesis of N-cyclopentyl-2-methylpyrazolo[1,5-a]quinazolin-5-amine (Compound 8)

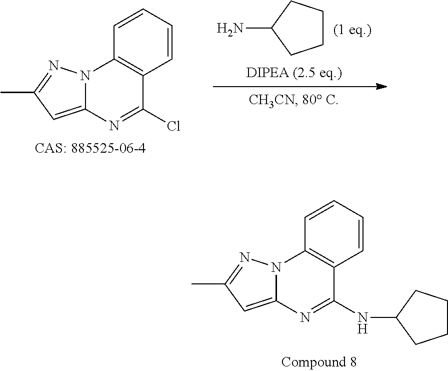 [SMILES: Cc1cc2nc(Cl)c3ccccc3n2n1 ; NC1CCCC1 ; Cc1cc2nc(NC3CCCC3)c3ccccc3n2n1]

S-Chloro-2-methylpyrazolo[1,5-a]quinazoline (CAS: 885525-06-4, 55 mg, 0.25 mmol), cyclopentylamine (22 mg, 0.25 mmol) and N,N-diisopropylethylamine (0.11 mL, 0.63 mmol) were combined in 5 mL of acetonitrile. The reaction was heated at 80° C. for 24 h. Next, the reaction was cooled and concentrated in vacuo. The resulting residue was taken up in CH₂Cl, washed with 2% acetic acid aqueous solution (2×), water, and brine. The organic layer was dried over Na₂SO₄, filtered and the organic solvent was evaporated. The resulting residue was purified by silica gel flash chromatography eluting with CH₂Cl₂: ethyl acetate to give N-cyclopentyl-2-methylpyrazolo[1,5-a]quinazolin-5-amine (ND-14445). ¹H NMR (400 MHZ, CDCl3) δ 8.30 (1H, dd, J=8.4, 0.6 Hz), 7.72 (1H, ddd, J=8.4, 7.3, 1.1 Hz), 7.61 (1H, dd, J=8.3, 1.0 Hz), 7.34 (1H, ddd, J=8.3, 7.3, 1.1 Hz), 6.05 (1H, s), 5.31 (1H, bd, J=6.1 Hz), 4.59 (1H, dquin, J=6.9, 6.9 Hz), 2.46 (3H, s), 2.28-2.17 (2H, m), 1.85-1.66 (4H, m), 1.61-1.50 (2H, m). ¹³C NMR (125 MHz, CDCl₃) δ 152.5, 151.7, 146.9, 136.6, 132.7, 123.4, 122.2, 115.3, 111.1, 94.6, 52.7, 33.4, 23.9, 14.6 Hz. HRMS (ESI-TOF, positive mode) m/z ([M+1]); Anal. Calcd for C₁₆H₁₉N₄ 267.161; Found 267.160.

### Example 4: Synthesis of N-cyclooctyl-2-methylpyrazolo[1,5-a]quinazolin-5-amine (Compound 9)

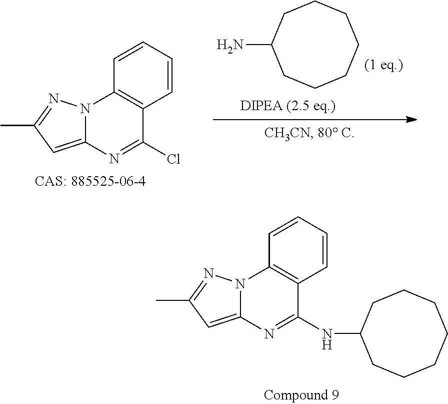 [SMILES: Cc1cc2nc(Cl)c3ccccc3n2n1 ; NC1CCCCCCC1 ; Cc1cc2nc(NC3CCCCCCC3)c3ccccc3n2n1]

5-Chloro-2-methylpyrazolo[1,5-a]quinazoline (CAS: 885525-06-4, 55 mg, 0.25 mmol), cyclooctylamine (32 mg, 0.25 mmol) and N,N-diisopropylethylamine (0.11 mL, 0.63 mmol) were combined in 5 mL of acetonitrile. The reaction was heated at 80° C. for 24 h. Next, the reaction was cooled and concentrated in vacuo. The resulting residue was taken up in CH₂Cl₂, washed with 2% acetic acid aqueous solution (2×), water, and brine. The organic layer was dried over Na₂SO₄, filtered and organic solvent was evaporated. The resulting residue was purified by silica gel flash chromatography eluting with CH₂Cl₂: ethyl acetate to give N-cyclooctyl-2-methylpyrazolo[1,5-a]quinazolin-5-amine (ND-14446). ¹H NMR (400 MHZ, CDCl3) δ 8.29 (1H, dd, J=8.4, 0.8 Hz), 7.72 (1H, ddd, J=8.4, 7.3, 1.1 Hz), 7.62 (1H, d, J=7.7 Hz), 7.34 (1H, ddd, J=8.3, 7.3, 1.1 Hz), 6.03 (1H, s), 5.29 (1H, bd, J=7.5 Hz), 4.49-4.38 (1H, m), 2.45 (3H, s), 2.09-1.98 (2H, m), 1.84-1.54 (12H, m). ¹³C NMR (125 MHz, CDCl₃) δ 152.5, 150.9, 147.0, 136.7, 132.7, 123.4, 122.1, 115.3, 111.2, 94.5, 50.4, 31.9, 27.4, 25.5, 23.7, 14.6 Hz. HRMS (ESI-TOF, positive mode) m/z ([M+1]); Anal. Calcd for C₁₉H₂₄N₄ 309.208; Found 309.213.

### Biological Assay

The compounds described and provided herein were evaluated for their potential to inhibit Cytochrome BD oxidase in Mycobacterium abscessus. M. abscessus ATCC 19977T wild-type (WT) and deficient for the expression of the cyt-bcc:aa₃ (ΔqcrCAB) were treated with a 2-fold dose-response (starting from 40 μM) of the drugs. ATP levels were measured after 6 to 15 hours of incubation using a commercial kit. Compounds targeting specifically the bd oxidase (BDO) to inhibit Oxidative phosphorylation (hence ATP levels) are expected to be active only in the ΔqcrCAB mutant strain.

Potencies of the tested compounds described and provided for herein in Mycobacterium abscessus are shown in Table 2. M. abscessus ATCC 19977T wild-type (WT) and deficient for the expression of the cyt-bcc:aa₃ (ΔqcrCAB) were treated with a 2-fold dose-response (starting from 40 μM) of the drugs. ATP levels were measured after 6 to 15 hours of incubation using a commercial kit. Compounds targeting specifically the BDO to inhibit Oxidative phosphorylation (hence ATP levels) are expected to be active only in the ΔqcrCAB mutant strain.

TABLE 2

ATP IC50 (μm)

| Compound | ΔgcrCAB | WT |
|---|---|---|
| 14 | >40 | >40 |
| 1 | 12.1 | >40 |
| 2 | >40 | >40 |
| 3 | 20 | >40 |
| 4 | 9.73 | >40 |
| 5 | 6.89 | >40 |
| 6 | >40 | >40 |
| 7 | >40 | >40 |
| 8 | 15.3 | >40 |
| 9 | 25.9 | >40 |
| 12 | 17.4 | >40 |
| 15A | 6.6 | >40 |
| 16A | 32.8 | >40 |
| 17 | 3.2 | >40 |
| 18 | 9.7 | >40 |
| 19A | 18.7 | >40 |
| 21 | 12.1 | >40 |
| 22 | 19.6 | >40 |
| 23 | 9 | >40 |
| 24 | 9.2 | >40 |
| 25 | 2.6 | >40 |
| 26 | 4.4 | >40 |

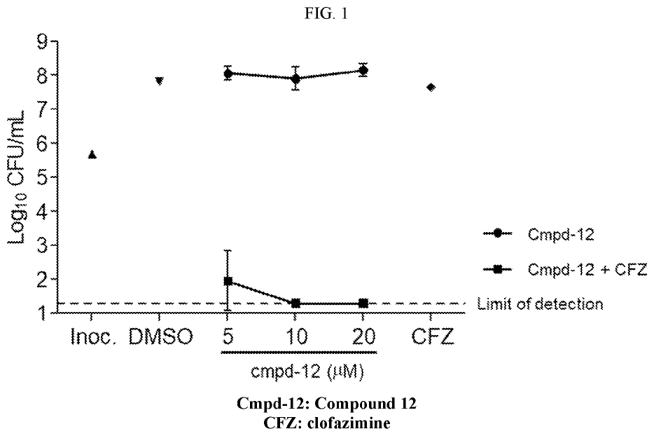

As shown in FIG. 1, it was demonstrated that Compound 12 synergizes with clofazimine to kill M. abscessus. This result is unexpected and surprising in that clofazimine is bactericidal in the presence of Compound 12, while clofazimine is bacteriostatic when tested alone. Hence, the combination of a BDO inhibitor with clofazimine is expected to be effective in treating mycobacterial infections.

NRF-NRFI06-2020-0004: National Research Foundation (NRF) Singapore under its Investigator ship Program is acknowledged.

While a number of embodiments of this disclosure have been described, it is apparent that the basic examples may be altered to provide other embodiments that utilize the compounds and methods of the present disclosure. Therefore, it will be appreciated that the scope of the present disclosure is to be defined by the appended claims rather than by the specific embodiments that have been represented by way of example.

What is claimed is:

1. A compound having a formula of: 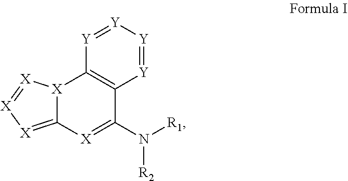
or a pharmaceutically acceptable salt thereof,
wherein:
each X is, independently, N or CR₃;
each Y is, independently, N or CR₄;
R₁, R₂, R₃, and R are each independently, H, D, halogen, R^{a}, —C(O)R^{a}, —CO₂R^{a}, —S(O)R^{a}, —SO₂R^{a}, —S(O)NHR^{a}, —SO₂NHR^{a}, —C(O)NHR^{a}, —N(R^{a})CO₂R^{a}, or N(R^{a})CONR^{a}₂;
each R^{a} is independently H, D, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; and provided that the compound is not

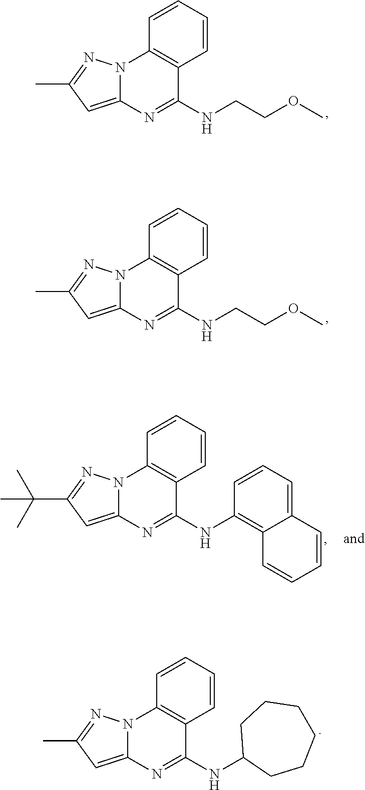

2. The compound of claim 1, wherein the compound has a formula of: 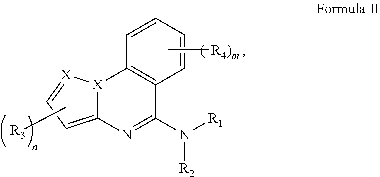
or a pharmaceutically acceptable salt thereof, wherein n is 0-2 and m is 0-4.

3. The compound according to claim 1 or 2, wherein R₂ is H.

4. The compound of claim 3, wherein the compound has a formula of: 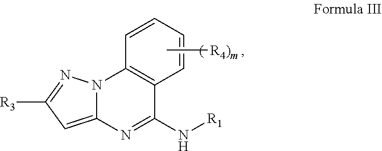
or a pharmaceutically acceptable salt thereof.

5. The compound of claim 4, wherein m is 1.

6. The compound of claim 5, wherein the compound has a formula of: 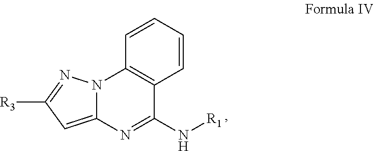
or a pharmaceutically acceptable salt thereof.

7. The compound of claim 6, wherein R₃ is H or optionally substituted C₁-C₆ alkyl, wherein C₁-C₆ alkyl is optionally substituted with one or more of CF₃, halogen, C₁-C₆ alkoxy, SF₃, and SF₅.

8. The compound of claim 7, wherein the compound has a formula of 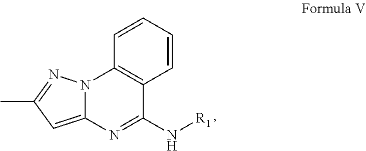
or a pharmaceutically acceptable salt thereof.

9. The compound of claim 8, wherein R₁ is optionally substituted C₃-C₂₂ cycloalkyl.

10. The compound of claim 9, wherein the compound has a formula of 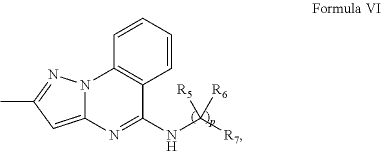
or a pharmaceutically acceptable salt thereof,
wherein:
R₅, R₆ and R₇ are each, independently, H, D, halogen, R^{b}, —C(O)R^{b}, —CO₂R^{b}, —S(O)R^{b}, SO₂R^{b}, —S(O)NHR^{b}, —SO₂NHR^{b}, —C(O)NHR^{b}, —N(R^{b})CO₂R^{b}, or N(R^{b})CONR^{b};
each R^{b} is independently H, D, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; and
p is 0-10.

11. The compound of claim 10, wherein p is 0.

12. The compound of claim 10, wherein p is 1.

13. The compound of claim 12, wherein the compound is 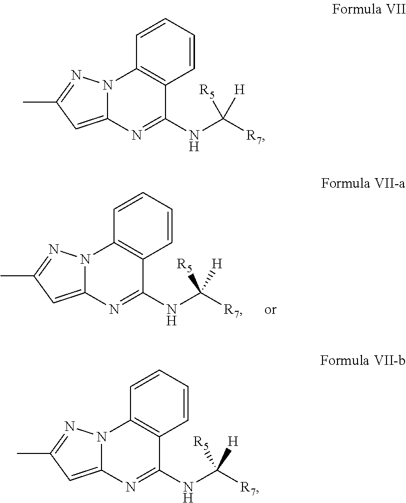
or a pharmaceutically acceptable salt thereof.

14. The compound of claim 13, wherein R₅ is H, CH₃, CO₂H, or CO₂Me.

15. The compound of any one of claims 10-14, wherein R₇ is C₃-C₂₂ cycloalkyl.

16. The compound of claim 15, wherein R₇ is 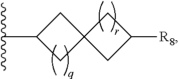
wherein:
q is 0-8;
r is 0-8;
R₈ is H, D, halogen, R^{c}, —C(O)R^{c}, —CO₂R^{c}, —S(O)R^{c}, SO₂R^{c}, —S(O)NHR^{c}, —SO₂NHR^{c}, —C(O)NHR^{c}, —N(R^{c})CO₂R^{c}, or N(R^{c})CONR^{c}; and
each R^{c} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl.

17. The compound of claim 16, wherein R₇ is

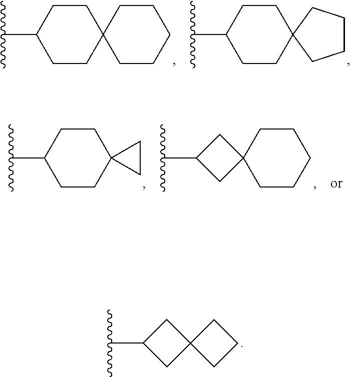

18. The compound of claim 15, wherein R₇ is 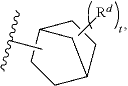
wherein:
R^{d} is H, D, halogen, R^{e}, —C(O)R^{e}, —CO₂R^{e}, —S(O)R^{e}, SO₂R^{e}, —S(O)NHR^{e}, —SO₂NHR^{e}, —C(O)NHR^{e}, —N(R^{e})CO₂R^{e}, or N(R^{e})CONR^{e};
each R^{e} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; and
t is 0-12.

19. The compound of claim 18, wherein R, is

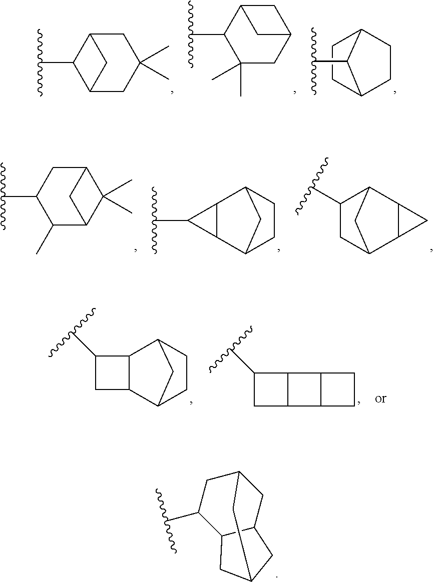

20. The compound of claim 15, wherein R₇ is 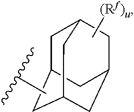
wherein:
R^{f} is H, D, halogen, R⁸, —C(O)R^{g}, —CO₂R^{g}, —S(O)R^{g}, SO₂R^{g}, —S(O)NHR^{g}, —SO₂NHR^{g}, —C(O)NHR^{g}, —N(R^{g})CO₂R^{g}, or N(R^{g})CONR^{g};
each R^{g} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; and
u is 0-12.

21. The compound of claim 15, wherein R₇ is 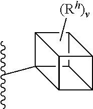
wherein:
R^{h} is H, D, halogen, R^{j}, —C(O)R^{j}, —CO₂R^{j}, —S(O)R^{j}, SO₂R^{j}, —S(O)NHR^{j}, —SO₂NHR^{j}, —C(O)NHR^{j}, —N(R^{j})CO₂R^{j}, or N(R^{j})CONR^{j};
each R^{j} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; and
v is 0-7.

22. The compound of claim 18, wherein v is 1.

23. The compound of claim 19, wherein R₇ is

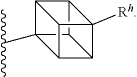

24. The compound of claim 20, wherein R^{h} is H, F, CN, NH₂, COOH, SO₂Cl, CH₂NH₂, CH₂OH, CH₂COOH, C≡CH, CH₂COOMe,

25. The compound of claim 15, wherein R₇ is 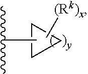
wherein:
R^{k} is H, D, halogen, R^{m}, —C(O)R^{m}, —CO₂R^{m}, —S(O)R^{m}, SO₂R^{m}, —S(O)NHR^{m}, —SO₂NHR^{m}, —C(O)NHR^{m}, —N(R^{m})CO₂R^{m}, or N(R^{m})CONR^{m};
each R^{m} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl;
y is 1-12; and
x is 0-12.

26. The compound of claim 25, wherein the compound has a formula of 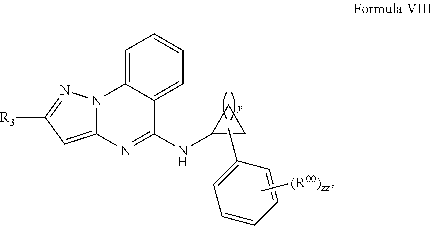
or a pharmaceutically acceptable salt thereof, wherein R₃ is H or optionally substituted C₁-C₆ alkyl, y is 0-6, R⁰⁰ is halo or haloalkyl, and zz is 0-5.

27. The compound of claim 25, wherein y is 4 or 5.

28. The compound of any one of claims 25-27, wherein R^{k} is H.

29. The compound of any one of claims 25-28, wherein x is 2.

30. The compound of claim 29, wherein R^{k} is F.

31. The compound of any one of claims 25-28, wherein x is 4.

32. The compound of claim 31, wherein R^{k} is Me.

33. The compound of claim 10, wherein p is 0-3.

34. The compound of claim 33, wherein R₇ is 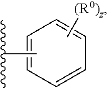
wherein:
R^{o} is H, D, halogen, R^{p}, —C(O)R^{p}, —CO₂R^{p}, —S(O)R^{p}, SO₂R^{p}, —S(O)NHR^{p}, —SO₂NHR^{p}, —C(O)NHR^{p}, —N(R^{p})CO₂R^{p}, or N(R^{p})CONR^{p};
each R^{p} is independently H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; and
z is 0-5.

35. The compound of claim 34, wherein R^{o} is H, D, CF₃, OCF₃, CN, or NR^{q}₂, wherein each R^{q} is, independently, H, D, CF₃, OCF₃, CN, SF₃, SF₅, halogen, optionally substituted C₁-C₆ alkyl, C₁-C₆ alkoxyl, C₁-C₆ haloalkyl, C₃-C₂₂ cycloalkyl, C₄-C₁₀ heterocycle, C₆-C₁₀ aryl, or C₅-C₉ heteroaryl; or two R^{q} together form a C₄-C₁₀ heterocycle.

36. The compound of claim 11, wherein Ry is optionally substituted C₁-C₆ alkyl.

37. The compound of claim 36, wherein R₇ is

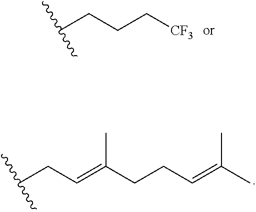 [SMILES: *CCCC(F)(F)F ; *C/C=C(\C)CCC=C(C)C]

38. The compound of claim 1, wherein the compound has a formula of 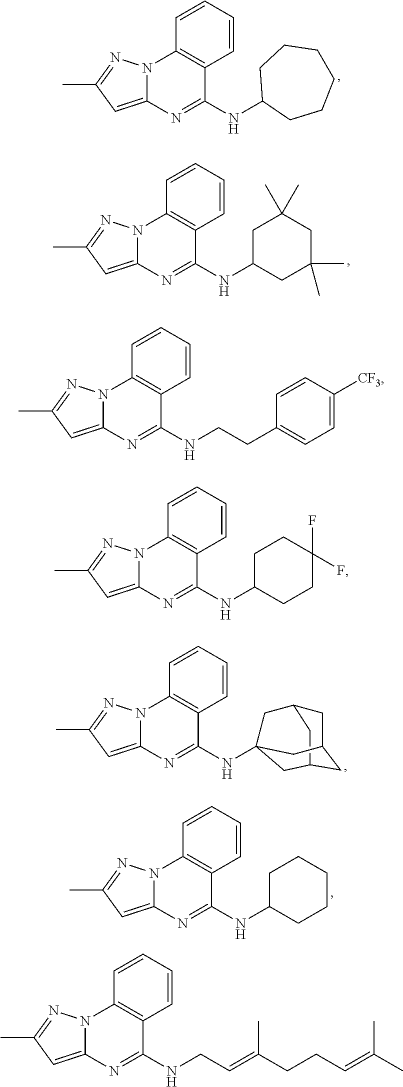 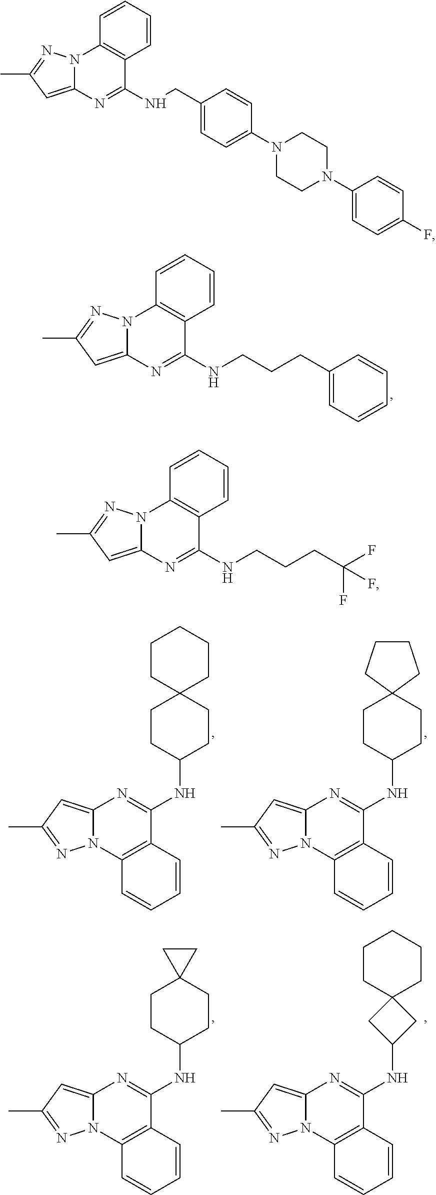 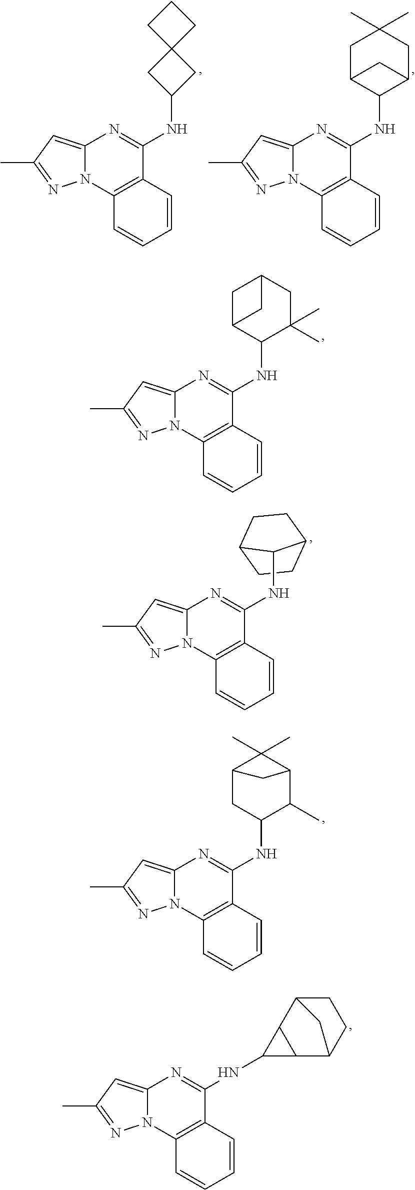 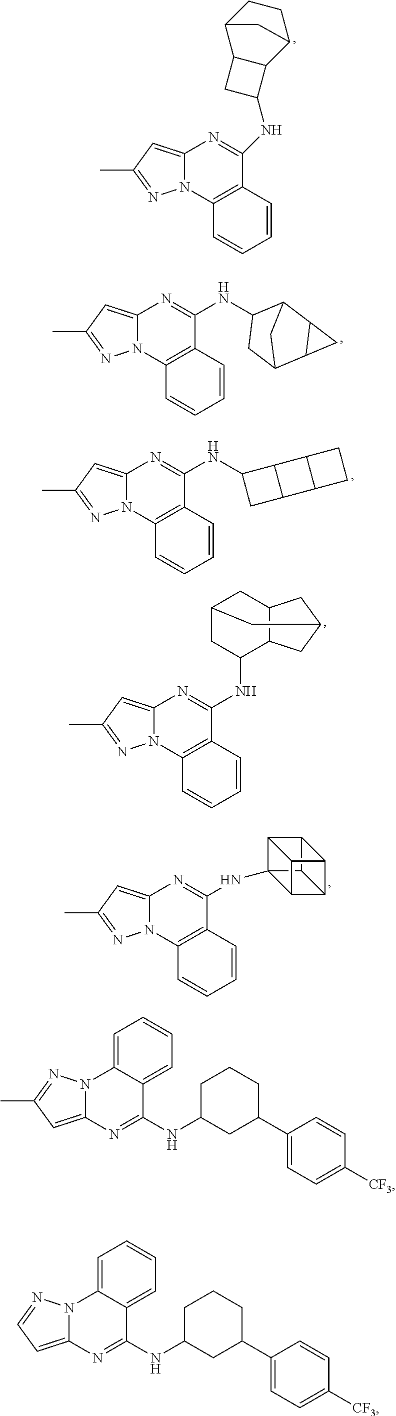 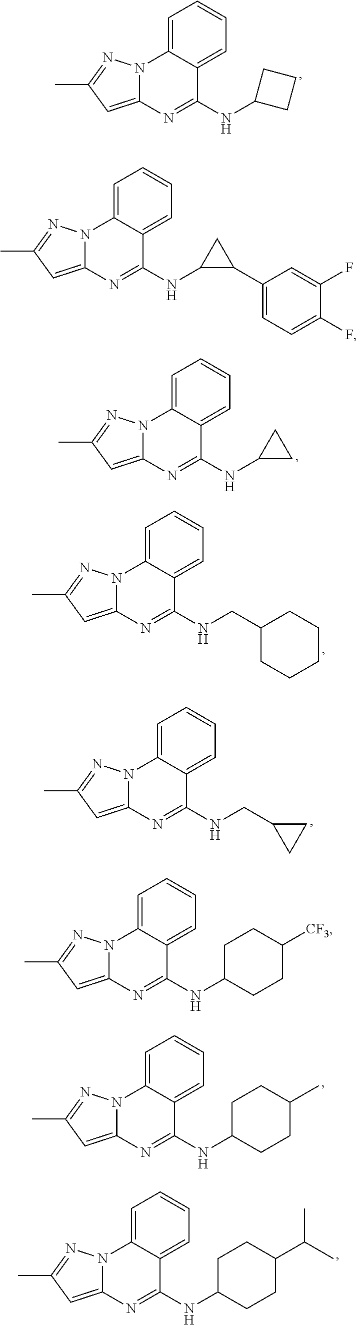 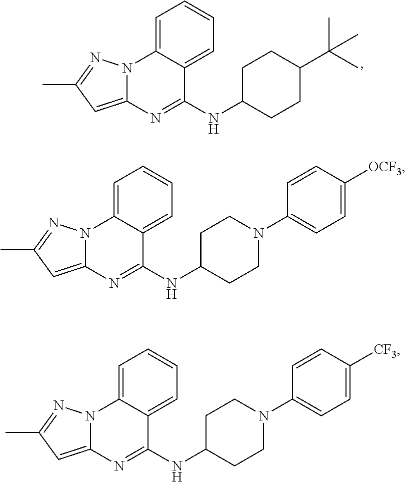
or a pharmaceutically acceptable salt thereof.

39. A pharmaceutical composition comprising the compound of any one of claims 1-38, or a pharmaceutically acceptable salt thereof.

40. A pharmaceutical composition comprising a compound having a formula of 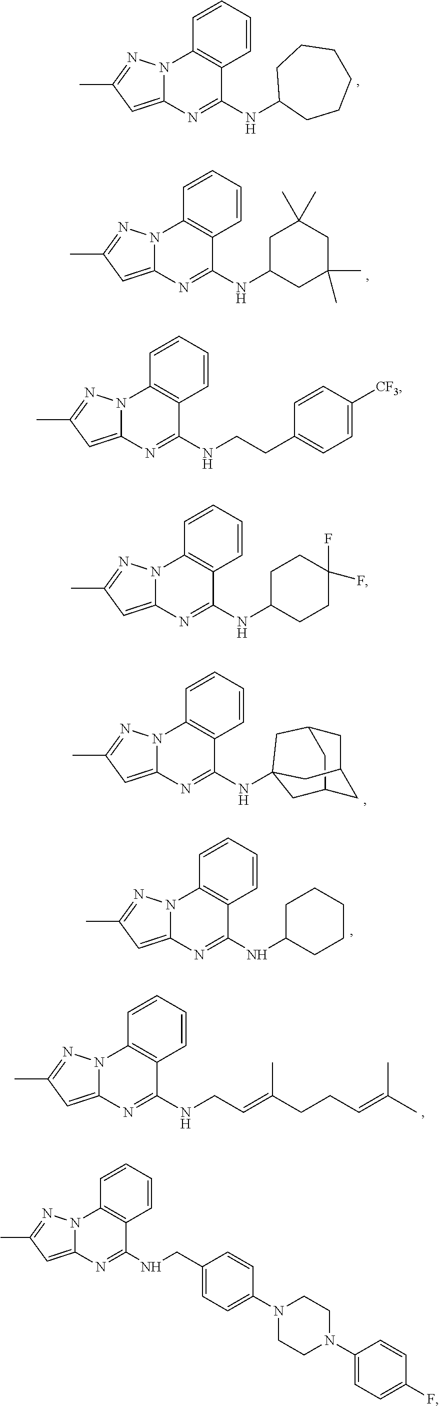 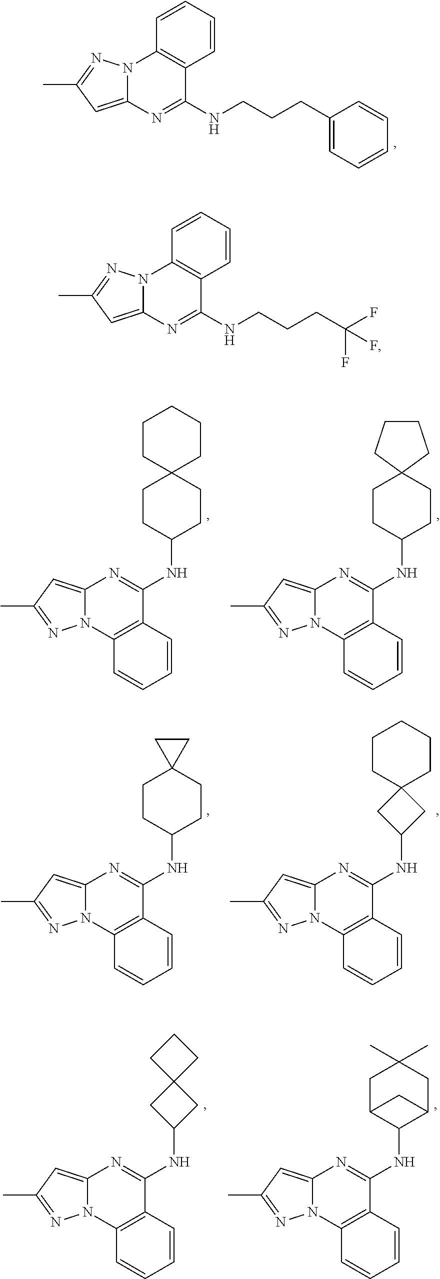 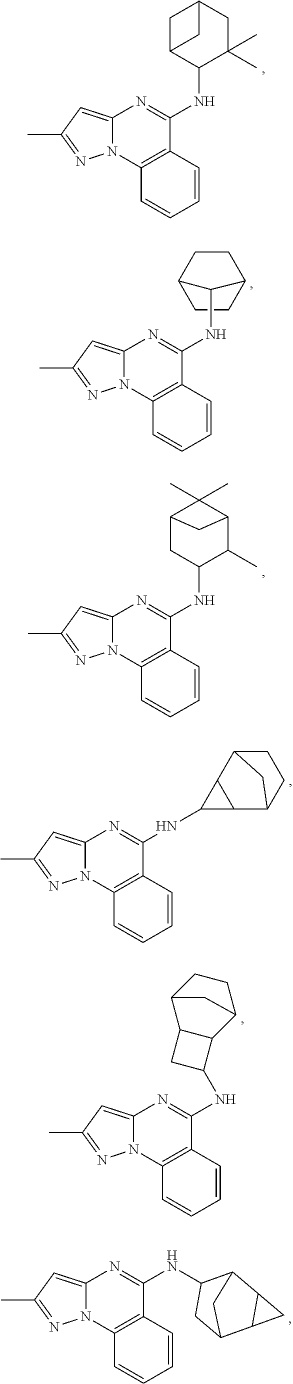 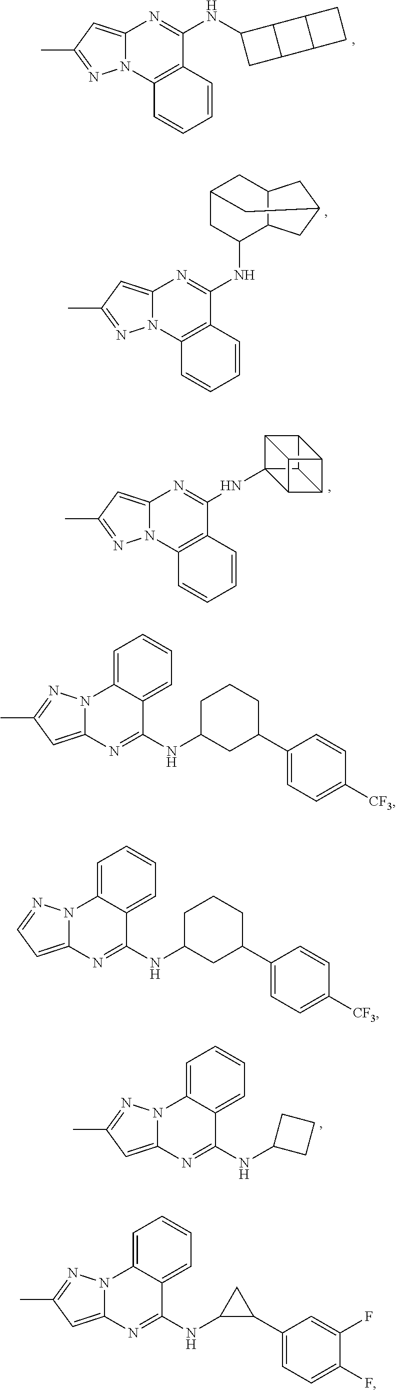 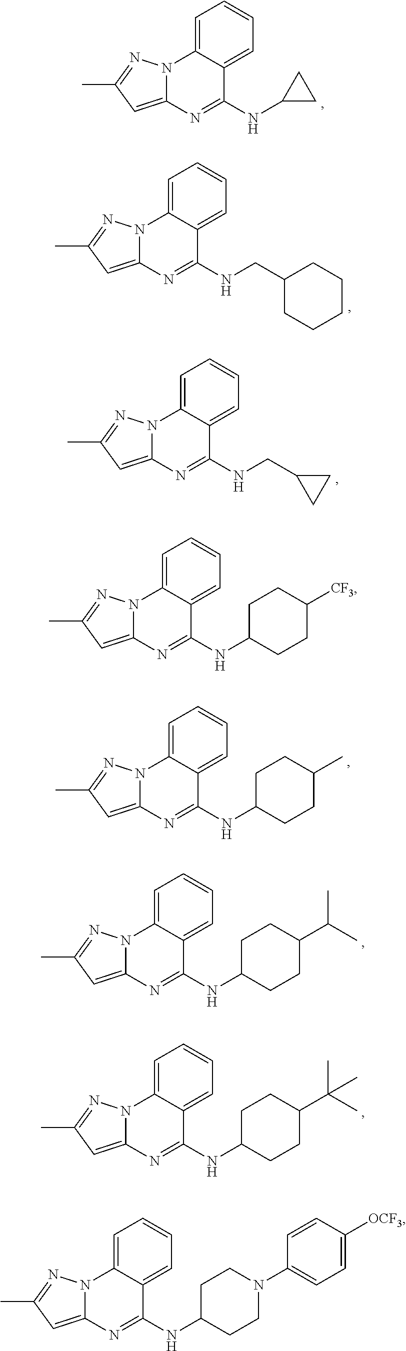 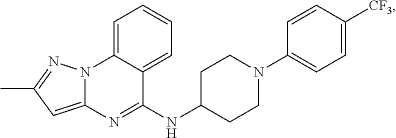 [SMILES: Cc1cc2nc(NC3CCN(c4ccc(C(F)(F)F)cc4)CC3)c3ccccc3n2n1]
or a pharmaceutically acceptable salt thereof.

41. The pharmaceutical composition according to claim 39 or 40, further comprising an inhibitor of the oxidative phosphorylation processes in mycobacteria.

42. The pharmaceutical composition of claim 41, wherein the inhibitor of the oxidative phosphorylation processes in mycobacteria is a QcrB inhibitor.

43. The pharmaceutical composition of claim 42, wherein the QcrB inhibitor is in the imidazopyridine class

44. The pharmaceutical composition of claim 42, wherein the QcrB inhibitor is Q203 or clofazimine or the like.

45. The pharmaceutical composition of any one of claims 39-44, further comprising a pharmaceutically acceptable carrier, adjuvant, or vehicle.

46. A method for treating a mycobacterial infection in a subject, comprising administering to the subject the compound of any one of claims 1-38 or a pharmaceutically acceptable salt thereof.

47. A method for treating a mycobacterial infection in a subject, comprising administering to the subject a compound having a formula of 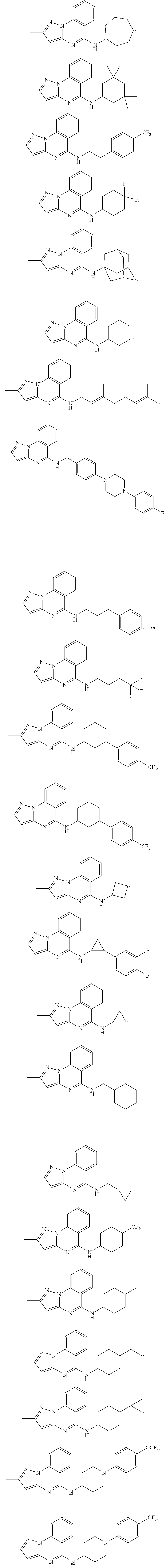
or a pharmaceutically acceptable salt thereof.

48. The method according to claim 46 or 47, wherein the compound or the pharmaceutically acceptable salt thereof, is present in a therapeutically effective amount.

49. The method of any one of claims 46-48, further comprising administering an inhibitor of the oxidative phosphorylation processes in mycobacteria.

50. The method of claim 49, wherein the inhibitor of the oxidative phosphorylation processes in mycobacteria is a QcrB inhibitor.

51. The method of claim 50, wherein the QcrB inhibitor is in the imidazopyridine class

52. The method of claim 51, wherein the QcrB inhibitor is Q203 or clofazimine or the like.

53. The method of any one of claims 49-52, wherein there is a synergistic effect between the compound and the inhibitor.

54. The method of claim 53, wherein the compound or the pharmaceutically acceptable salt thereof, is administered in a synergistic therapeutically effective amount.

55. The method according to claim 53 or 54, wherein the inhibitor or the pharmaceutically acceptable salt thereof, is administered in a synergistic therapeutically effective amount.

56. A method for treating a mycobacterial infection in a subject, comprising administering to the subject the pharmaceutical composition of any one of claims 39-40.

57. A method for treating a mycobacterial infection in a subject, comprising administering to the subject the pharmaceutical composition of any one of claims 41-45.

58. The method according to claim 56 or 57, wherein the compound or the pharmaceutically acceptable salt thereof, is present in a therapeutically effective amount.

59. The method according to claim 57 or 58, wherein there is a synergistic effect between the compound and the inhibitor.

60. The method of claim 59, wherein the compound or the pharmaceutically acceptable salt thereof, is present in a synergistic therapeutically effective amount.

61. The method accordingly to claim 59 or 60, wherein the inhibitor or the pharmaceutically acceptable salt thereof, is present in a synergistic therapeutically effective amount.

62. The method of any one of claims 46-61, wherein the mycobacterial infection is caused by a bacteria from the Mycobacterium tuberculosis complex.

63. The method of any one of claims 46-61, wherein the mycobacterial infection is caused by a non-tuberculosis mycobacteria (NTM) such as those belonging to the Mycobacterium abscessus complex or to the Mycobacterium avium complex.

64. The method of any one of claims 46-63, wherein the subject is in need thereof.

65. The compound of claim 38, the pharmaceutical composition of claim 40, or the method of claim 47, wherein the compound has the formula of 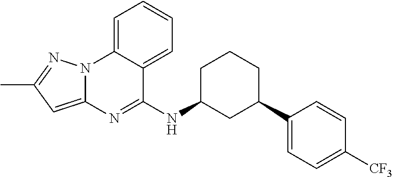 [SMILES: Cc1cc2nc(N[C@H]3CCC[C@@H](c4ccc(C(F)(F)F)cc4)C3)c3ccccc3n2n1]
